(19) United States
(12) Patent Application Publication
(10) Pub. No.: US 2024/0148790 A1
(43) Pub. Date: May 9, 2024

(54) EXPEDITED ADMINISTRATION OF ENGINEERED LYMPHOCYTES

(71) Applicant: Kite Pharma, Inc., Foster City, CA (US)

(72) Inventors: Zhenhuan Hu, New Berlin, WI (US); Hairong Xu, Calabasas, CA (US); Sachin Vadgama, Chesham (GB)

(21) Appl. No.: 18/495,499

(22) Filed: Oct. 26, 2023

Related U.S. Application Data
(60) Provisional application No. 63/506,288, filed on Jun. 5, 2023.
(60) Provisional application No. 63/386,831, filed on Dec. 9, 2022.
(60) Provisional application No. 63/381,507, filed on Oct. 28, 2022.

(51) Int. Cl. A61K 35/17; A61K 39/00; A61K 45/06; A61P 7/00; A61P 35/00; C12N 5/0783; C12N 15/86; G16H 20/17

(52) U.S. Cl. CPC A61K 35/17; A61K 39/4611; A61K 39/4631; A61K 39/4632; A61K 45/06; A61P 7/00; A61P 35/00; C12N 5/0636; C12N 15/86; G16H 20/17; A61K 2239/38; C12N 2740/10043

(57) ABSTRACT

Provided herein are methods for expedited manufacturing of engineered lymphocytes which are demonstrated to be associated with a favorable complete response rate and overall survival, and reduced risk of prolonged thrombocytopenia. The expedited manufacturing process can prepare transduced lymphocytes having improved efficacy or reduced adverse effects in treating cancer. An example process includes acquiring lymphocytes from a patient through apheresis, incubating the lymphocytes with a polynucleotide vector to transduce the lymphocytes to produce transduced lymphocytes, culturing the transduced lymphocytes, and infusing the transduced lymphocytes to the patient predicting a likelihood of a complete response, an overall survival rate, and a risk of prolonged thrombocytopenia in a subject receiving an immunotherapy.

### CROSS REFERENCE TO RELATED APPLICATIONS

This application claims priority to U.S. Provisional Patent Application No. 63/381,507, filed on Oct. 28, 2022, U.S. Provisional Patent Application No. 63/386,831, filed on Dec. 9, 2022, and U.S. Provisional Patent Application No. 63/506,288, filed on Jun. 5, 2023, each of which is hereby incorporated in its entirety.

### SEQUENCE LISTING

The instant application contains a Sequence Listing which has been submitted electronically in XML file format and is hereby incorporated by reference in its entirety. Said XML copy, created on Oct. 11, 2023, is named K-1143-US_NP_SL.xml and is 28,791 bytes in size.

### BACKGROUND

Chimeric antigen receptors (CARs) and engineered T cell receptors (TCRs) include binding domains capable of interacting with a particular tumor antigen. Such binding capability allows the immune cells to target and kill cancer cells. A significant challenge comes with the highly complicated and lengthy autologous cell engineering and production process. During the process, lymphocytes collected from the patients must be shipped to the process center, while the produced cells have to be cryopreserved and then shipped back to the patient for implantation. This highly complicated process necessarily leads to high costs and limited clinical applications. Therefore, there is a strong unmet need to develop processes that are shorter in duration to improve patient outcomes.

### SUMMARY

Provided herein are methods for expedited manufacturing and administration of engineered lymphocytes, which are demonstrated to be associated with a favorable complete response rate and overall survival, and reduced risk of prolonged thrombocytopenia. The expedited manufacturing process, in particular, having a reduced vein-to-vein time, is associated with improved efficacy or reduced adverse effects in treating cancer. Also provided herein are methods predicting a likelihood of a complete response, an overall survival rate, and a risk of prolonged thrombocytopenia in a subject receiving an immunotherapy.

An embodiment of the disclosure is related to a method for preparing lymphocytes having improved efficacy and/or reduced adverse effects in treating cancer, the method comprising acquiring lymphocytes from a patient through apheresis; incubating the lymphocytes with a polynucleotide vector to transduce the lymphocytes to produce transduced lymphocytes; culturing the transduced lymphocytes to obtain a sample of cultured lymphocytes; and infusing the sample to the patient, wherein the time taken from acquiring the lymphocytes to infusing the sample is not longer than 28 days.

In an embodiment of the disclosure, the patient has a greater than 55% likelihood of having complete response; a greater than 45% likelihood of having overall survival at 24 months; and/or a lower than 30% likelihood of developing prolonged thrombocytopenia.

In an embodiment of the disclosure, the time taken from acquiring the lymphocytes to infusing the sample is not longer than 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, or 6 days.

In an embodiment of the disclosure, the method further comprises administering lymphodepleting chemotherapy, wherein the lymphodepleting chemotherapy is administered within 5 days, 4 days, 3 days, 2 days, or 1 days of the infusing step.

An embodiment of the disclosure is related to a method for preventing and/or reducing the likelihood of prolonged thrombocytopenia in a patient having r/r LBCL, the method comprising acquiring lymphocytes from the patient through apheresis; incubating lymphocytes with a polynucleotide vector to transduce the lymphocytes to produce transduced lymphocytes; culturing the transduced lymphocytes to obtain a sample of cultured lymphocytes; and infusing the sample to the patient, wherein the time taken from acquiring the lymphocytes to infusing the sample is not longer than 28 days.

An embodiment of the disclosure is related to a method for predicting a likelihood of a complete response in a patient to an immunotherapy, comprising: determining a period of time from a leukapheresis step of said patient to an administration of said immunotherapy to said patient; grouping said patient into one of a plurality of groups based on said determining a period of time, said plurality of groups comprising: a first group characterized by a period of time of up to 28 days from said leukapheresis step to said administration of said immunotherapy to said patient; a second group characterized by a period of time between 28 days to 40 days from said leukapheresis step to said administration of said immunotherapy to said patient; and a third group characterized by a period of time of at least 40 days from said leukapheresis step to said administration of said immunotherapy to said patient; and determining said likelihood of a complete response in said patient based at least in part on which of said plurality of groups said patient is grouped into, wherein said patient has at least about a 55% likelihood of a complete response if said patient is grouped within said first group or said second group, and wherein said patient has at least about a 42% likelihood of a complete response if said patient is grouped within said third group.

In some embodiments of the disclosure, said patient has about a 60% likelihood of a complete response if said patient is grouped within said first group or said second group.

An embodiment of the disclosure relates to a method for predicting an overall survival rate in a patient to an immunotherapy, comprising: determining a period of time from a leukapheresis step of said patient to an administration of said immunotherapy to said patient; grouping said patient into one of a plurality of groups based on said determining a period of time, said plurality of groups comprising: a first group characterized by a period of time of up to 28 days from said leukapheresis step to said administration of said immunotherapy to said patient; a second group characterized by a period of time of between 28 days to 40 days from said leukapheresis step to said administration of said immunotherapy to said patient; and a third group characterized by a period of time of at least 40 days from said leukapheresis step to said administration of said immunotherapy to said patient; and determining said overall survival rate in said patient based at least in part on which of said plurality of groups said patient is grouped into, wherein said patient has at least about a 49% overall survival rate if said patient is grouped within said first group, wherein said patient has at least about a 48% overall survival rate if said patient is grouped within said second group, and wherein said patient has at least about a 30% overall survival rate if said patient is grouped within said third group.

An embodiment of the disclosure relates to a method for predicting a risk of thrombocytopenia in a patient receiving an immunotherapy, comprising: determining a period of time from a leukapheresis step of said patient to an administration of said immunotherapy to said patient; grouping said patient into one of a plurality of groups based on said determining a period of time, said plurality of groups comprising: a first group characterized by a period of time of up to 28 days from said leukapheresis step to said administration of said immunotherapy to said patient; a second group characterized by a period of time of between 28 days to 40 days from said leukapheresis step to said administration of said immunotherapy to said patient; and a third group characterized by a period of time of at least 40 days from said leukapheresis step to said administration of said immunotherapy to said patient; and determining said risk of thrombocytopenia in said patient based at least in part on which of said plurality of groups said patient is grouped into, wherein said patient has about an 18% risk of thrombocytopenia if said patient is grouped within said first group, wherein said patient has about a 25% risk of thrombocytopenia if said patient is grouped within said second group, and wherein said patient has about a 34% risk of thrombocytopenia if said patient is grouped within said third group.

An embodiment of the disclosure relates to a method for predicting a life expectancy and quality-adjusted life years in a patient that has received an immunotherapy, comprising: determining a period of time from a leukapheresis step of said patient to an administration of said immunotherapy to said patient, wherein the period of time is a short period or a long period; assigning a probability of successful infusion based on the period of time; entering the patient information into a survival model to determine the life expectancy and the quality-adjusted life years of the patient.

In an embodiment of the disclosure, the immunotherapy comprises one or more CARs recognizing one or more tumor antigens.

In an embodiment of the disclosure, the immunotherapy is axicabtagene ciloleucel or brexucabtagene autoleucel.

In an embodiment of the disclosure, the patient has relapsed or refractory (r/r) large B-cell lymphoma (LBCL).

### DETAILED DESCRIPTION

### Definitions

In order for the present disclosure to be more readily understood, certain terms are first defined below. Additional definitions for the following terms and other terms are set forth throughout the Specification.

Unless specifically stated or obvious from context, as used herein, the term “or” is understood to be inclusive and covers both “or” and “and”.

The term “and/or” where used herein is to be taken as specific disclosure of each of the two specified features or components with or without the other. Thus, the term “and/or” as used in a phrase such as “A and/or B” herein is intended to include A and B; A or B; A (alone); and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless specifically stated or evident from context the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within one or more than one standard deviation per the practice in the art. “About” or “comprising essentially of” can mean a range of up to 10% (i.e., ±10%). Thus, “about” can be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg can include any amount between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “comprising essentially of” should be assumed to be within an acceptable error range for that particular value or composition.

“Administering” refers to the physical introduction of an agent to a subject, such as a modified T cell disclosed herein, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The term “allogeneic” refers to any material derived from one individual which is then introduced to another individual of the same species.

The term “antibody” (Ab) includes, without limitation, a glycoprotein immunoglobulin which binds specifically to an antigen. In general, and antibody can comprise at least two heavy (H) chains and two light (L) chains interconnected by disulfide bonds, or an antigen-binding molecule thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system. In general, human antibodies are approximately 150 kD tetrameric agents composed of two identical heavy (H) chain polypeptides (about 50 kD each) and two identical light (L) chain polypeptides (about 25 kD each) that associate with each other into what is commonly referred to as a “Y-shaped” structure. The heavy and light chains are linked or connected to one another by a single disulfide bond; two other disulfide bonds connect the heavy chain hinge regions to one another, so that the dimers are connected to one another and the tetramer is formed. Naturally-produced antibodies are also glycosylated, e.g., on the CH2 domain.

An “antigen binding molecule,” “antigen binding portion,” “antigen binding fragment,” or “antibody fragment” refers to any molecule that comprises the antigen binding parts (e.g., CDRs) of the antibody from which the molecule is derived. An antigen binding molecule can include the antigenic complementarity determining regions (CDRs). Examples of antibody fragments include, but are not limited to, Fab, Fab′, F(ab′)2, and Fv fragments, dAb, linear antibodies, scFv antibodies, and multispecific antibodies formed from antigen binding molecules. Peptibodies (i.e., Fc fusion molecules comprising peptide binding domains) are another example of suitable antigen binding molecule. In some embodiments, the antigen binding molecule binds to an antigen on a tumor cell. In some embodiments, the antigen binding molecule binds to an antigen on a cell involved in a hyperproliferative disease or to a viral or bacterial antigen. In certain embodiments an antigen binding molecule is a chimeric antigen receptor (CAR) or an engineered T cell receptor (TCR).

The term “variable region” or “variable domain” is used interchangeably. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VL” and “VL domain” are used interchangeably to refer to the light chain variable region of an antibody or an antigen-binding molecule thereof.

The terms “VH” and “VH domain” are used interchangeably to refer to the heavy chain variable region of an antibody or an antigen-binding molecule thereof.

A number of definitions of the CDRs are commonly in use: Kabat numbering, Chothia numbering, AbM numbering, or contact numbering. The AbM definition is a compromise between the two used by Oxford Molecular's AbM antibody modelling software. The contact definition is based on an analysis of the available complex crystal structures.

The term “autologous” refers to any material derived from the same individual to which it is later to be re-introduced. For example, the engineered autologous cell therapy (eACT™) method described herein involves collection of lymphocytes from a patient, which are then engineered to express, e.g., a CAR construct, and then administered back to the same patient.

“Chimeric antigen receptor” or “CAR” refers to a molecule engineered to comprise a binding motif and a means of activating immune cells (for example T cells such as naive T cells, central memory T cells, effector memory T cells or combination thereof) upon antigen binding. CARs are also known as artificial T cell receptors, chimeric T cell receptors or chimeric immunoreceptors. In some embodiments, a CAR comprises a binding motif, an extracellular domain, a transmembrane domain, one or more co-stimulatory domains, and an intracellular signaling domain. A T cell that has been genetically engineered to express a chimeric antigen receptor may be referred to as a CAR T cell. “Extracellular domain” (or “ECD”) refers to a portion of a polypeptide that, when the polypeptide is present in a cell membrane, is understood to reside outside of the cell membrane, in the extracellular space.

A “T cell receptor” or “TCR” refers to antigen-recognition molecules present on the surface of T cells. During normal T cell development, each of the four TCR genes, a, 3, 7, and 6, may rearrange leading to highly diverse TCR proteins.

The term “heterologous” means from any source other than naturally occurring sequences. For example, a heterologous sequence included as a part of a costimulatory protein is amino acids that do not naturally occur as, i.e., do not align with, the wild type human costimulatory protein. For example, a heterologous nucleotide sequence refers to a nucleotide sequence other than that of the wild type human costimulatory protein-encoding sequence.

Term “identity” refers to the overall relatedness between polymeric molecules, e.g., between nucleic acid molecules (e.g., DNA molecules and/or RNA molecules) and/or between polypeptide molecules. Methods for the calculation of a percent identity as between two provided polypeptide sequences are known. Calculation of the percent identity of two nucleic acid or polypeptide sequences, for example, may be performed by aligning the two sequences for optimal comparison purposes (e.g., gaps may be introduced in one or both of a first and a second sequences for optimal alignment and non-identical sequences may be disregarded for comparison purposes). The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, optionally taking into account the number of gaps, and the length of each gap, which may need to be introduced for optimal alignment of the two sequences. Comparison or alignment of sequences and determination of percent identity between two sequences may be accomplished using a mathematical algorithm, such as BLAST (basic local alignment search tool). In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical (e.g., 85-90%, 85-95%, 85-100%, 90-95%, 90-100%, or 95-100%).

The immune cells of the immunotherapy can come from any source known in the art. For example, immune cells can be differentiated in vitro from a hematopoietic stem cell population, or immune cells can be obtained from a subject. Immune cells can be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the immune cells can be derived from one or more immune cell lines available in the art. Immune cells can also be obtained from a unit of blood collected from a subject using any number of techniques known to the skilled artisan, such as FICOLL™ separation and/or apheresis. Additional methods of isolating immune cells for an immune cell therapy are disclosed in U.S. Patent Publication No. 2013/0287748, which is herein incorporated by references in its entirety.

A “patient” includes any human who is afflicted with a cancer (e.g., a lymphoma or a leukemia). The terms “subject” and “patient” are used interchangeably herein.

The term “pharmaceutically acceptable” refers to a molecule or composition that, when administered to a recipient, is not deleterious to the recipient thereof, or that any deleterious effect is outweighed by a benefit to the recipient thereof. With respect to a carrier, diluent, or excipient used to formulate a composition as disclosed herein, a pharmaceutically acceptable carrier, diluent, or excipient must be compatible with the other ingredients of the composition and not deleterious to the recipient thereof, or any deleterious effect must be outweighed by a benefit to the recipient. The term “pharmaceutically acceptable carrier” means a pharmaceutically-acceptable material, composition or vehicle, such as a liquid or solid filler, diluent, excipient, or solvent encapsulating material, involved in carrying or transporting an agent from one portion of the body to another (e.g., from one organ to another). Each carrier present in a pharmaceutical composition must be “acceptable” in the sense of being compatible with the other ingredients of the formulation and not deleterious to the patient, or any deleterious effect must be outweighed by a benefit to the recipient. Some examples of materials which may serve as pharmaceutically acceptable carriers comprise: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

The term “pharmaceutical composition” refers to a composition in which an active agent is formulated together with one or more pharmaceutically acceptable carriers. In some embodiments, the active agent is present in a unit dose amount appropriate for administration in a therapeutic regimen that shows a statistically significant probability of achieving a predetermined therapeutic effect when administered to a relevant subject or population. In some embodiments, a pharmaceutical composition may be formulated for administration in solid or liquid form, comprising, without limitation, a form adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

The terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions.

The term “reference” describes a standard or control relative to which a comparison is performed. For example, in some embodiments, an agent, animal, individual, population, sample, sequence, or value of interest is compared with a reference or control that is an agent, animal, individual, population, sample, sequence, or value. In some embodiments, a reference or control is tested, measured, and/or determined substantially simultaneously with the testing, measuring, or determination of interest. In some embodiments, a reference or control is a historical reference or control, optionally embodied in a tangible medium. Generally, a reference or control is determined or characterized under comparable conditions or circumstances to those under assessment. When sufficient similarities are present to justify reliance on and/or comparison to a selected reference or control.

A “therapeutically effective amount,” “effective dose,” “effective amount,” or “therapeutically effective dosage” of a therapeutic agent, e.g., engineered CAR T cells, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The terms “transduction” and “transduced” refer to the process whereby foreign nucleic acid is introduced into a cell via viral vector (see Jones et al., “Genetics: principles and analysis,” Boston: Jones & Bartlett Publ. (1998)). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In one embodiment, “treatment” or “treating” includes a partial remission. In another embodiment, “treatment” or “treating” includes a complete remission. In some embodiments, treatment may be of a subject who does not exhibit signs of the relevant disease, disorder and/or condition and/or of a subject who exhibits only early signs of the disease, disorder, and/or condition. In some embodiments, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

The term “vector” refers to a recipient nucleic acid molecule modified to comprise or incorporate a provided nucleic acid sequence. One type of vector is a “plasmid,” which refers to a circular double stranded DNA molecule into which additional DNA may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) may be integrated into the genome of a host cell upon introduction into the host cell, and thereby are replicated along with the host genome. Moreover, certain vectors comprise sequences that direct expression of inserted genes to which they are operatively linked. Such vectors may be referred to herein as “expression vectors.” Standard techniques may be used for engineering of vectors, e.g., as found in Sambrook et al., Molecular Cloning: A Laboratory Manual (2d ed., Cold Spring Harbor Laboratory Press, Cold Spring Harbor, N.Y. (1989)), which is incorporated herein by reference.

An embodiment of the disclosure relates to a method for preparing lymphocytes having improved efficacy and/or reduced adverse effects in treating cancer, the method including: acquiring lymphocytes from a patient through apheresis; incubating the lymphocytes with a polynucleotide vector to transduce the lymphocytes to produce transduced lymphocytes; culturing the transduced lymphocytes to obtain a sample of cultured lymphocytes; and infusing the sample to the patient. In such an embodiment, the time taken from acquiring the lymphocytes to infusing the sample is not longer than 28 days.

An embodiment of the disclosure relates to a method for preventing and/or reducing the likelihood of prolonged thrombocytopenia in a patient having r/r LBCL, the method including: acquiring lymphocytes from the patient through apheresis; incubating lymphocytes with a polynucleotide vector to transduce the lymphocytes to produce transduced lymphocytes; culturing the transduced lymphocytes to obtain a sample of cultured lymphocytes; and infusing the sample to the patient. In such an embodiment, the time taken from acquiring the lymphocytes to infusing the sample is not longer than 28 days.

An embodiment of the disclosure relates to any of the methods discussed above, where the patient has a greater than 55% likelihood of having complete response; a greater than 45% likelihood of having overall survival at 24 months; and/or a lower than 30% likelihood of developing prolonged thrombocytopenia.

An embodiment of the disclosure relates to any of the methods discussed above, where the time taken from acquiring the lymphocytes to infusing the sample is not longer than 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, or 6 days.

An embodiment of the disclosure relates to any of the methods discussed above, further including administering lymphodepleting chemotherapy, and where the lymphodepleting chemotherapy is administered within 5 days, 4 days, 3 days, 2 days, or 1 days of the infusing step.

An embodiment of the disclosure relates to any of the methods discussed above, and does not include cryopreservation of the cultured lymphocytes.

An embodiment of the disclosure relates to any of the methods discussed above, where the transduced lymphocytes are cultured for less than 72 hours, 48 hours or 36 hours.

An embodiment of the disclosure relates to any of the methods discussed above, where the incubation is carried out in a closed system.

An embodiment of the disclosure relates to any of the methods discussed above, where the closed system has an inner surface area of at least 1500 cm².

An embodiment of the disclosure relates to any of the methods discussed above, where the closed system has an inner surface coated with a recombinant human fibronectin, and the coating is carried out with a solution that includes about 1-10 μg/ml of the recombinant human fibronectin.

An embodiment of the disclosure relates to any of the methods discussed above, where the inner surface is further in contact with a second solution including the polynucleotide vector, and where the second solution has a volume of about 200 mL.

An embodiment of the disclosure relates to any of the methods discussed above, where the coating further includes a drain of the second solution.

An embodiment of the disclosure relates to any of the methods discussed above, where the sample in the closed system has at least 1.5×10⁸ lymphocytes.

An embodiment of the disclosure relates to any of the methods discussed above, where the sample has at least 4×10⁸ lymphocytes.

An embodiment of the disclosure relates to any of the methods discussed above, where the lymphocytes are peripheral blood mononuclear cells (PBMCs) or T cells.

An embodiment of the disclosure relates to any of the methods discussed above, where the sample includes CD4+ and CD8+ T cells.

An embodiment of the disclosure relates to any of the methods discussed above, where a total of 10,000 to 1,000,000 cultured lymphocytes per kilogram of the patient are administered to the patient.

An embodiment of the disclosure relates to any of the methods discussed above, where a total of 20,000 to 400,000 cultured lymphocytes per kilogram of the patient are administered to the patient.

An embodiment of the disclosure relates to any of the methods discussed above, where at least 15% of the cultured lymphocytes are transduced with the vector.

An embodiment of the disclosure relates to any of the methods discussed above, where the polynucleotide vector is a viral vector.

An embodiment of the disclosure relates to any of the methods discussed above, where the viral vector is a retroviral vector or a lentiviral vector.

An embodiment of the disclosure relates to any of the methods discussed above, where the vector encodes one or more chimeric antigen receptors (CAR) or one or more T cell receptors (TCR).

An embodiment of the disclosure relates to any of the methods discussed above, where the one or more CARs has an intracellular costimulatory domain.

An embodiment of the disclosure relates to any of the methods discussed above, where the intracellular costimulatory domain is a signaling region of a protein selected from the group consisting of DAP-10, CD28, OX-40, 4-1BB (CD137), CD2, CD7, CD27, CD30, CD40, programmed death-1 (PD-1), inducible T cell costimulator (ICOS), lymphocyte function-associated antigen-1 (LFA-1, CD11a/CD18), CD3 gamma, CD3 delta, CD3 epsilon, CD247, CD276 (B7-H3), tumor necrosis factor superfamily member 14, TNFSF14, LIGHT), NKG2C, Ig alpha (CD79a), Fc gamma receptor, MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, CDS, GITR, BAFFR, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, IA4, CD49D, ITGA6, VLA-6, CD49f, ITGAD (CD11d), ITGAE (CD103), ITGAL (CD11a), ITGAM (CD11b), ITGAX (CD11c), ITGB1, CD29, ITGB2, CD18, ITGB7, NKG2D, TNFR2, TRANCE (RANKL), DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMFI, CD150, IPO-3), BLAME (SLAMF8), SELPLG (CD162), LTBR, LAT, GADS, SLP-76, PAG (Cbp), CD19a, a ligand that specifically binds with CD83, and combinations thereof.

An embodiment of the disclosure relates to any of the methods discussed above, where the intracellular costimulatory domain is a signaling region of CD28.

An embodiment of the disclosure relates to any of the methods discussed above, where the one or more CARs recognizes one or more tumor antigens.

An embodiment of the disclosure relates to any of the methods discussed above, where the tumor antigen is CD19.

An embodiment of the disclosure relates to any of the methods discussed above, where the lymphocyte that includes the CAR is axicabtagene ciloleucel or brexucabtagene autoleucel.

An embodiment of the disclosure relates to any of the methods discussed above, where the patient has relapsed or refractory (r/r) large B-cell lymphoma (LBCL).

An embodiment of the disclosure relates to any of the methods discussed above, where the tumor antigen is CD19 and CD20.

An embodiment of the disclosure relates a method for predicting a likelihood of a complete response in a patient to an immunotherapy, the method including: determining a period of time from a leukapheresis step of the patient to an administration of the immunotherapy to the patient; grouping the patient into one of a plurality of groups based on the determining a period of time, the plurality of groups including: a first group characterized by a period of time of up to 28 days from the leukapheresis step to the administration of the immunotherapy to the patient; a second group characterized by a period of time of between 28 days to 40 days from the leukapheresis step to the administration of the immunotherapy to the patient; and a third group characterized by a period of time of at least 40 days from the leukapheresis step to the administration of the immunotherapy to the patient; and determining the likelihood of a complete response in the patient based at least in part on which of the plurality of groups the patient is grouped into. In such an embodiment, the patient has at least about a 55% likelihood of a complete response if the patient is grouped within the first group or the second group, and the patient has at least about a 42% likelihood of a complete response if the patient is grouped within the third group.

An embodiment of the disclosure relates the method for predicting a likelihood of a complete response in a patient to an immunotherapy discussed above, where the patient has about a 60% likelihood of a complete response if the patient is grouped within the first group or the second group.

An embodiment of the disclosure relates the method for predicting a likelihood of a complete response in a patient to an immunotherapy discussed above, where the immunotherapy includes one or more CARs recognizing one or more tumor antigens.

An embodiment of the disclosure relates the method for predicting a likelihood of a complete response in a patient to an immunotherapy discussed above, where the tumor antigen is CD19.

An embodiment of the disclosure relates the method for predicting a likelihood of a complete response in a patient to an immunotherapy discussed above, where the tumor antigen is CD19 and CD20.

An embodiment of the disclosure relates the method for predicting a likelihood of a complete response in a patient to an immunotherapy discussed above, where the immunotherapy is axicabtagene ciloleucel or brexucabtagene autoleucel.

An embodiment of the disclosure relates the method for predicting a likelihood of a complete response in a patient to an immunotherapy discussed above, where the patient has relapsed or refractory (r/r) large B-cell lymphoma (LBCL).

An embodiment of the disclosure relates the method a method for predicting an overall survival rate in a patient to an immunotherapy, the method including: determining a period of time from a leukapheresis step of the patient to an administration of the immunotherapy to the patient; grouping the patient into one of a plurality of groups based on the determining a period of time, the plurality of groups including: a first group characterized by a period of time of up to 28 days from the leukapheresis step to the administration of the immunotherapy to the patient; a second group characterized by a period of time of between 28 days to 40 days from the leukapheresis step to the administration of the immunotherapy to the patient; and a third group characterized by a period of time of at least 40 days from the leukapheresis step to the administration of the immunotherapy to the patient; and determining the overall survival rate in the patient based at least in part on which of the plurality of groups the patient is grouped into. In such an embodiment, the patient has at least about a 49% overall survival rate if the patient is grouped within the first group, the patient has at least about a 48% overall survival rate if the patient is grouped within the second group, and the patient has at least about a 30% overall survival rate if the patient is grouped within the third group.

An embodiment of the disclosure relates the method for predicting an overall survival rate in a patient to an immunotherapy discussed above, where the immunotherapy includes one or more CARs recognizing one or more tumor antigen.

An embodiment of the disclosure relates the method for predicting an overall survival rate in a patient to an immunotherapy discussed above, where the tumor antigen is CD19.

An embodiment of the disclosure relates the method for predicting an overall survival rate in a patient to an immunotherapy discussed above, where the tumor antigen is CD19 and CD20.

An embodiment of the disclosure relates the method for predicting an overall survival rate in a patient to an immunotherapy discussed above, where the immunotherapy is axicabtagene ciloleucel or brexucabtagene autoleucel.

An embodiment of the disclosure relates the method for predicting an overall survival rate in a patient to an immunotherapy discussed above, where the patient has relapsed or refractory (r/r) large B-cell lymphoma (LBCL).

An embodiment of the disclosure relates to a method for predicting a risk of thrombocytopenia in a patient receiving an immunotherapy, the method including: determining a period of time from a leukapheresis step of the patient to an administration of the immunotherapy to the patient; grouping the patient into one of a plurality of groups based on the determining a period of time, the plurality of groups including: a first group characterized by a period of time of up to 28 days from the leukapheresis step to the administration of the immunotherapy to the patient; a second group characterized by a period of time of between 28 days to 40 days from the leukapheresis step to the administration of the immunotherapy to the patient; and a third group characterized by a period of time of at least 40 days from the leukapheresis step to the administration of the immunotherapy to the patient; and determining the risk of thrombocytopenia in the patient based at least in part on which of the plurality of groups the patient is grouped into.

In such an embodiment, the patient has about an 18% risk of thrombocytopenia if the patient is grouped within the first group, the patient has about a 25% risk of thrombocytopenia if the patient is grouped within the second group, and the patient has about a 34% risk of thrombocytopenia if the patient is grouped within the third group.

An embodiment of the disclosure relates to the method for predicting a risk of thrombocytopenia in a patient receiving an immunotherapy discussed above, where the immunotherapy includes one or more CARs recognizing one or more tumor antigens.

An embodiment of the disclosure relates to the method for predicting a risk of thrombocytopenia in a patient receiving an immunotherapy discussed above, where the tumor antigen is CD19.

An embodiment of the disclosure relates to the method for predicting a risk of thrombocytopenia in a patient receiving an immunotherapy discussed above, where the tumor antigen is CD19 and CD20.

An embodiment of the disclosure relates to the method for predicting a risk of thrombocytopenia in a patient receiving an immunotherapy discussed above, where the immunotherapy is axicabtagene ciloleucel or brexucabtagene autoleucel.

An embodiment of the disclosure relates to the method for predicting a risk of thrombocytopenia in a patient receiving an immunotherapy discussed above, where the patient has relapsed or refractory (r/r) large B-cell lymphoma (LBCL).

An embodiment of the disclosure relates to a method for predicting life expectancy and a quality-adjusted life years in a patient that has received an immunotherapy, the method including: determining a period of time from a leukapheresis step of the patient to an administration of the immunotherapy to the patient, where the period of time is a short period or a long period; assigning a probability of successful infusion based on the period of time; entering the patient information into a survival model to determine the life expectancy and the quality-adjusted life years of the patient.

An embodiment of the disclosure relates to the method for predicting life expectancy and a quality-adjusted life years in a patient that has received an immunotherapy discussed above, where the short period is about 24 days or less.

An embodiment of the disclosure relates to the method for predicting life expectancy and a quality-adjusted life years in a patient that has received an immunotherapy discussed above, where the long period is about 54 days or more.

An embodiment of the disclosure relates to the method for predicting life expectancy and a quality-adjusted life years in a patient that has received an immunotherapy discussed above, where the long period is about 37 days or more.

An embodiment of the disclosure relates to the method for predicting life expectancy and a quality-adjusted life years in a patient that has received an immunotherapy discussed above, where the short period of time indicates a gain in the life expectancy and a gain in the quality-adjusted life years of the patient to be greater than 5 years and where the long period of time indicates the gain in the life expectancy and the gain in the quality-adjusted life years of the patient to be less than 5 years.

An embodiment of the disclosure relates to the method for predicting life expectancy and a quality-adjusted life years in a patient that has received an immunotherapy discussed above, where the immunotherapy includes one or more CARs recognizing one or more tumor antigens.

An embodiment of the disclosure relates to the method for predicting life expectancy and a quality-adjusted life years in a patient that has received an immunotherapy discussed above, where the tumor antigen is CD19.

An embodiment of the disclosure relates to the method for predicting life expectancy and a quality-adjusted life years in a patient that has received an immunotherapy discussed above, where the tumor antigen is CD19 and CD20.

An embodiment of the disclosure relates to the method for predicting life expectancy and a quality-adjusted life years in a patient that has received an immunotherapy discussed above, where the immunotherapy is axicabtagene ciloleucel or brexucabtagene autoleucel.

An embodiment of the disclosure relates to the method for predicting life expectancy and a quality-adjusted life years in a patient that has received an immunotherapy discussed above, where the patient has relapsed or refractory (r/r) large B-cell lymphoma (LBCL).

### Expedited Manufacturing of Engineered Lymphocytes

A typical autologous CAR-T manufacturing process begins with leukapheresis. A patient's apheresis material is collected at a treatment center. This process takes approximately 3-4 hours, during which time about 10 to 20 liters of blood is recirculated and approximately 100-500 mL of apheresis material is collected. The apheresis material collection bag is shipped cold to a central manufacturing facility.

From the apheresis material, T cells are enriched and then are transduced with a retroviral vector containing the CAR gene. The transduced cells are expanded in culture until they reach the target dose, at which point the cells are washed and cryopreserved for shipment back to the treatment center. Each lot undergoes a series of tests to ensure it meets certain standards prior to release.

Once the treatment center receives the CAR-T cell product from the manufacturing facility, the patient undergoes lymphodepleting (LD) chemotherapy. Lymphodepletion for axicabtagene ciloleucel consists of a regimen of fludarabine (30 mg/m²) and cyclophosphamide (500 mg/m²) for 3 days (day −5, day −4, and day −3). Currently, centers usually admit patients prior to certain CAR-T cell infusions. The CAR-T cells are administered 3 days after completion of lymphodepletion (day 0) and infused at a target dose of, e.g., 2×10⁶ CAR-T cells/kg for axicabtagene ciloleucel.

The entire process from apheresis to infusion (vein-to-vein) may take a median time of 28 days for axicabtagene ciloleucel, 45 day for tisagenlecleucel, and 37 days for lisocabtagene maraleucel.

Vein-to-vein time refers to the time from leukapheresis to infusion. The vein-to-vein time can be impacted by factors including, but not limited to, the time from leukapheresis to delivery of the product to an authorized treatment center, the time from leukapheresis to product shipment, transportation time, the time from leukapheresis to product release from the manufacturing site, and the time it takes to manufacture the product.

Shorter vein-to-vein time, as Example 1 demonstrates, is associated with improved complete response (CR) rate and overall survival (OS), and reduced risks of prolonged thrombocytopenia. Such findings, therefore, underscore the importance of development and deployment of expedited CAR-T manufacturing processes.

Without limitation, in one embodiment, the vein-to-vein time may be reduced by shortening the time needed for transportation. The time for transportation of the apheresis material to the manufacturing facility and the transduced cells back to the treatment center, in some embodiments, may be reduced by using faster shipping, or using a manufacturing facility that is closer to the treatment center.

In another embodiment, the vein-to-vein time may be reduced with better coordination which helps to eliminate the requirement for cryopreservation. For instance, if the patient is ready for the infusion when the cells are ready, then no cryopreservation may be required.

In another embodiment, the capability of manufacturing products that meet specification requirements (in-specification) may reduce vein-to-vein time.

The vein-to-vein time may also depend on the quality and quantity of the starting apheresis material. When the starting apheresis material includes fewer or lower-quality lymphocytes, longer vein-to-vein time may be needed. In view of the improved performance associated with shorter vein-to-vein time, therefore, the time for expanding the lymphocytes may be reduced even if that may result in a reduced number of transduced cells for infusion.

An important portion of the manufacturing process includes lymphocyte transduction and expansion. Through years' improvements, a transduction/expansion process that takes 7 days (following the enrichment step) has been developed, as described below. On top of these successes, a further refined process that can be completed within 5 or even 3 days (counting following an enrichment step) has been developed and is also described below. As described herein, the 5-day process includes transduction preparation and implementation steps with a higher number of lymphocytes in contact with vectors immobilized to recombinant fibronectin coated to the inner surface of a closed system. Such an improved transduction procedure allows a much-abbreviated post-transduction cell expansion step. Transduced cells prepared from the 5-day process skewed towards more juvenile cells which led to better in vivo antitumor efficacy.

In some embodiments, when the post-transduction expansion is eliminated, the transduction and expansion (following the enrichment step) procedure can be completed within only 3 days. Compared to the 5-day process, this 3-day process generates a population of cells with a greater percentage of juvenile cells and decreased percentage of more mature, differentiated and activated, cells. Accordingly, not only did the cell products from the 3-day process exhibit the best in vivo antitumor efficacy, such greatly improved efficacy could even be achieved with much lower doses.

Example processes for a 7 day, a 5 day, and a 3 day lymphocyte manufacture process are described below.

### Seven-Day Lymphocyte Manufacturing Process

In some embodiments, the therapeutic cellular product is produced following a 7-day process of preparing lymphocytes that are transduced with a polynucleotide vector, such a viral vector, that encodes a therapeutic protein. The prepared lymphocytes can be useful for treating various diseases such as cancer, especially when the therapeutic protein is a chimeric antigen receptor (CAR) or T cell receptor (TCR) designed to target a cancer cell.

As used throughout, the terms “7-day process” and “7-day lymphocyte manufacturing process” are used interchangeably and refer to a CAR cell manufacturing process which takes about 7 days following initial enrichment and activation steps. The 7-day process is at least 8 days in length from the initial enrichment and activation steps to a harvesting step, and can be between 8 to 11 days in total when including the enrichment and activation steps.

Apheresis Collection.

White blood cells can be collected (leukapheresis) using standard apheresis equipment, such as Cobe® Spectra, Spectra Optia®, Fenwal™ Amicus® or equivalent. The leukapheresis process typically yielded approximately 200-400 mL of an apheresis product from patients. The apheresis product can be subjected to the manufacturing process on-site, or optionally shipped at 1-10° C. to a facility to undergo the manufacturing process in a different location. Further process steps can be conducted in an ISO 7 cell culture process suite (or similar clean room type environment).

Volume Reduction.

Where appropriate, a volume reduction step can be performed using a cell processing instrument such as the Sepax® 2 laboratory instrument (Biosafe SA; Houston, TX) or equivalent, and carried out using a standard aseptic tubing kit. Given the variability in the number of cells and volume of incoming source material from each subject (approximately 200-400 mL), the volume reduction step is designed to standardize the volume of cells to approximately 120 mL. In the event that the apheresis volume is less than 120 mL, the volume reduction step need not be performed, and the cells directly carried to the lymphocyte enrichment step. The volume reduction step can standardize the volume of cells received from each subject, retain mononuclear cells, achieve consistent cell yield and high cell viability, and maintain a closed system to minimize risk of contamination.

Lymphocyte Enrichment.

Following the Volume Reduction step, the cells can be subjected to Ficoll based separation on a cell processing instrument, such as the Sepax® 2 or equivalent, using the separation protocol developed and recommended by the instrument manufacturer (NeatCell Program) and using a standard aseptic tubing kit. The lymphocyte enrichment step reduces product related impurities such as RBCs, and granulocytes, enriches and concentrates the mononuclear cells, washes and reduces process related residuals such as Ficoll, and formulates the cells in growth media in preparation for cell activation, as well as achieving consistent cell yield and high cell viability. The closed system minimizes environmental contamination.

The process may be carried out in an ISO 7 area at ambient temperature and all connections may be conducted either using a sterile tubing welder, or carried out in an ISO 5 laminar flow hood.

Lymphocyte Activation. The Lymphocyte Activation step can be carried out either with freshly processed cells from the Lymphocyte Enrichment, or previously cryopreserved cells. In the event that cryopreserved cells are used, the cells may be thawed using developed protocols prior to use.

The Lymphocyte Activation step selectively activates lymphocytes to become receptive to retroviral vector transduction, reduces the viable population of all other cell types, achieves consistent cell yield and high lymphocyte viability, and maintains a closed system to minimize the risk of contamination. Lymphocyte Activation can be achieved with lymphocyte stimulating agents such as anti-CD3 antibodies and IL-2. In some embodiments, the Lymphocyte Activation step occurs within a closed system having an inner surface area of at least 700 cm².

Wash 1.

Following the Lymphocyte Activation step, the cells can be washed using cell processing equipment, such as the Sepax® 2 or equivalent, with fresh culture media in a standard aseptic kit using developed protocols by the manufacturer. The cells were optionally concentrated to a final volume of approximately 100 mL in preparation for retroviral vector transduction. The Wash 1 step reduces process related residuals such as anti-CD3 antibody, spent growth media, and cellular debris; achieves consistent cell yield and high T cell viability, maintains a closed system to minimize the risk of contamination; and concentrates and delivers a sufficient number of viable T cells in a small volume appropriate for initiation of transduction.

Transduction.

Activated cells from the Wash 1 step in of fresh cell growth media can be transferred to a cell culture bag (Origen Biomedical PL240 or comparable) which has been previously prepared by first coating the bags with a recombinant fibronectin or fragments thereof such as RetroNectin® (Takara Bio, Japan), and subsequently incubated with retroviral vector according to defined procedures prior to introduction of the activated cells. RetroNectin® coating (10 μg/mL) can be carried out at a temperature of 2-8° C. for 20±4 hr, washed with dilute buffer, and subsequently incubated with thawed retroviral vector for approximately 180-210 min at 37±1° C. and 5±0.5% CO₂. After the addition of cells to the bag, the transduction can be carried out for 20±4 hr at 37±1° C. and 5±0.5% CO₂. The retroviral transduction step cultures the activated T cells in the presence of the retroviral vector under controlled conditions in order to allow for efficient transduction to take place, achieves consistent cell yield and high cell viability, and maintains a closed system in order to minimize the risk of contamination.

Wash 2.

Following the retroviral transduction step, the cells can be washed with fresh growth media using cell processing equipment, such as the Sepax® 2 or equivalent, in a standard aseptic kit using protocols developed by the manufacturer, and the cells were concentrated to a final volume of approximately 100 mL in preparation for the expansion step. The Wash 2 step can reduce process related residuals such as retroviral vector particles, vector production process residuals, spent growth media, and cellular debris achieve consistent cell yield and high cell viability; maintain a closed system to minimize the risk of contamination; and exchange spent growth media for fresh media with a target number of cells in a specified volume appropriate for initiation of expansion step.

Lymphocyte Expansion.

Cells from the Wash 2 step can be aseptically transferred to a culture bag (Origen Biomedical PL325 or equivalent) and diluted with fresh cell growth media and cultured for approximately 72 hr at 37±1° C. and 5±0.5% CO₂. The cell density was measured daily starting on Day 5. Because doubling times of the T cells may vary slightly from subject to subject, additional growth time beyond 72 hr (i.e., 3-6 days) may be necessary in the event that the total cell number is insufficient to deliver a target dose of CAR-positive T cells/kg of subject weight. The lymphocyte expansion step is designed to culture the cells under controlled conditions in order to produce a sufficient number of transduced cells for delivering an efficacious dose, maintain a closed system to minimize risk of contamination, and achieve consistent cell yield and high cell viability. One such efficacious dose or target dose includes 2×10⁶ FMC63-28Z CAR positive or FMC63-CD828BBZ CAR positive T cells/kg (±20%) of subject weight that were produced via transduction with either the MSGV-FMC63-28Z retroviral vector or the MSGV-FMC63-CD828BBZ retroviral vector, respectively, both of which are described in detail in Kochenderfer et al., J Immunother. 2009 September; 32(7): 689-702.

Wash 3 and Concentrate.

Following the lymphocyte expansion step, the cells can be washed with 0.9% saline using a cell processing instrument, such as the Sepax® 2 or equivalent, in a standard aseptic kit using developed protocols by the manufacturer, and the cells were concentrated to a final volume of approximately 35 mL in preparation for the formulation and cryopreservation. The wash 3 step is designed to reduce process related residuals such as retroviral production process residuals, spent growth media, and cellular debris; achieve consistent cell yield and high cell viability; and maintain a closed system to minimize risk of contamination.

Once the cells have been concentrated and washed into 0.9% saline, an appropriate cell dose may be formulated for preparation of the final cryopreserved product.

The embodiments described herein provide for efficient production of an engineered lymphocyte therapy within 7 days.

### Five-Day Lymphocyte Manufacturing Process

In some embodiments, the therapeutic cellular product is produced following a 5-day process of preparing lymphocytes that are transduced with a polynucleotide vector, such a viral vector, that encodes a therapeutic protein. The prepared lymphocytes can be useful for treating various diseases such as cancer, especially when the therapeutic protein is a chimeric antigen receptor (CAR) or T cell receptor (TCR) designed to target a cancer cell. Such a 5-day process is based on the 7-day process described above.

As used throughout, the terms “5-day process” and “5-day lymphocyte manufacturing process” are used interchangeably and refer to a CAR cell manufacturing process which takes about 5 days following initial enrichment and activation steps. The 5-day process is 6 days in length from the initial enrichment and activation steps to a harvesting step, and can be between 6 to 9 days in total when including the enrichment and activation steps.

During the 7-day process, lymphocytes are enriched and activated on day 0; transduction bag is coated with recombinant fibronectin on day 1; viral transduction is conducted on day 2; transduced lymphocytes are washed and then expanded on days 3 and 4; expansion continues with media changed each day on day 5 and 6; and the final cell products are harvested on day 7. From about 1.2×10⁹ lymphocytes acquired from apheresis, about 2.4×10⁸ lymphocytes are incubated with the viral vectors for transduction.

In the 5-day process, no change is made to the process on day 0. On days 1 and 2, however, a larger bag was used. Instead of the Origen Biomedical PL240 bag being used for transduction, the Origen Biomedical PL325 bag was used, or more preferably, a PL750 bag. The larger bag allowed a larger volume of the vector (200 mL instead of 100 mL) and more lymphocytes (between 3.2×10⁸ and 6×10⁸ instead of 2.4×10⁸) used in the transduction step.

Interestingly, the increased volume of transduction, along with more vectors and starting lymphocytes, did not result in unacceptable reduction of transduction efficiency (54% to 35.15%) or cell viability (92% to 92.4%). Accordingly, the cell expansion step, which required 4 days in the 7-day process, can be reduced to 2 days, enabling harvesting of the final cell products on day 5.

The modest reduction of transduction rate, however, did not correlate to clinical efficacy or patient safety. Also importantly, it was found that the cell products from the 5-day process included increased percentages of juvenile cells among both CD4+ and CD8+ T cell populations which are believed to be associated with improved therapeutic efficacy. These variations, it is noted, are within historical ranges of donor runs collected from the 7-day process.

The shortened 5-day process met specification requirements for transduction efficiency, therapeutic efficacy, and safety. Meanwhile, the 5-day products exhibited a more juvenile phenotype within the historical ranges.

### Three-Day Lymphocyte Manufacturing Process

In some embodiments, the therapeutic cellular product is produced following a 3-day process of preparing lymphocytes that are transduced with a polynucleotide vector, such a viral vector, that encodes a therapeutic protein. The prepared lymphocytes can be useful for treating various diseases such as cancer, especially when the therapeutic protein is a chimeric antigen receptor (CAR) or T cell receptor (TCR) designed to target a cancer cell. The 3-day process is based on the 7-day and 5-day processes described above.

As used throughout, the terms “3-day process” and “3-day lymphocyte manufacturing process” are used interchangeably and refer to a CAR cell manufacturing process which takes about 3 days from initial enrichment and activation steps. The 3-day process is about 4 days in length from the initial enrichment and activation step to a harvesting step. The 3-day process does not include a cell expansion step comprising one or more days following a transduction step and preceding a harvesting step.

In the 3-day process, the day 0-1 procedures are similar to the 5-day process, including the fibronectin-coating of a larger bag (e.g., Origen Biomedical PL325 or preferably PL750) for the subsequent transduction on day 2. On day 2, however, in some embodiments, only about 4.8×10⁸ lymphocytes can be used in the transduction step, with the same amount of viral vectors (200 mL). Alternatively, in some embodiments, about 6×10⁸ lymphocytes can be used in the transduction step, with 200 mL of viral vectors. Alternatively, in some embodiments, only about 4.8×10⁸ lymphocytes can be used in the transduction step, with the 100 mL of viral vectors. Another important difference is that, unlike in the 5-day and 7-day processes, a reduced step of T cell expansion is conducted. Instead, the transduced lymphocytes can be harvested on day 3, allowing the entire process to be completed within 3 days from the initial enrichment and activation steps.

Given the lack of a specific T cell expansion step in the 3-day process, the cell products harvested included a slightly smaller percentage of T cells (CD3+). Importantly, however, the 3-day products can include an even greater percentage of juvenile (naïve) T cells as compared to the 7-day process.

Even though not tested with lymphocytes from the same donors, the data also indicate that the naïve T cell percentages from the 3-day process were significantly higher than from the 5-day process. Within CD4+ T cells, the 3-day process generated about 55.75% naïve T cells while the 5-day process generated about 40.65%; within CD8+ T cells, the 3-day process generated about 37.35% naïve T cells while the 5-day process generated about 3.93%.

Put in other terms, the 3-day process can generate roughly a 1.4-fold increase in the percentage of CD4+ naïve T cells versus the percentage of such cells observed from the 5-day process, and roughly an 9.5-fold increase in the percentage of CD8+ naïve T cells versus the percentage of such cells observed from the 5-day process. Also, the 3-day process can generate roughly a 3.0-fold increase in the percentage of CD4+ naïve T cells versus the historical average of such cells observed from the 7-day process, and roughly an 18.0-fold increase in the percentage of CD8+ naïve T cells versus the historical average of such cells observed from the 7-day process.

Inversely, within CD4+ T cells, the 3-day process can generate only about 4.35% effector memory T cells while the 5-day process can generate about 8.55%; within CD8+ T cells, the 3-day process can only generate about 9.85% effector memory T cells while the 5-day process can generate about 15.85%.

Put in other terms, the 3-day process can generate roughly a 2.0-fold decrease in the percentage of CD4+ effector memory T cells versus the percentage of such cells observed from the 5-day process, and roughly a 3.6-fold decrease in the percentage of CD8+ effector memory T cells versus the percentage of such cells observed from the 5-day process. Also, the 3-day process can generate roughly a 6.0-fold decrease in the percentage of CD4+ effector memory T cells versus the historical average of such cells observed from the 7-day process, and roughly a 3.5-fold decrease in the percentage of CD8+ effector memory T cells versus the historical average of such cells observed from the 7-day process.

Cell viability measurements showed that, throughout the 3-day process, cell viability stayed high (>90%), while the wash step on day 2 caused the most reduction on cell viability. By contrast, the 5-day and 7-day processes included additional washing steps, each of which contributed to additional drop in cell viability.

In accordance with one embodiment of the present disclosure, therefore, provided is a method for preparing transduced lymphocytes having improved efficacy or reduced adverse effects in treating cancer. In some embodiments, the method entails acquiring lymphocytes from a patient through apheresis; incubating the lymphocytes with a polynucleotide vector to transduce the lymphocytes to produce transduced lymphocytes; culturing the transduced lymphocytes; and infusing the transduced lymphocytes to the patient. In some embodiments, the method entails shortening the manufacturing time of the therapeutic cellular product (i.e. transduced lymphocytes) by using any of the 7-day, 5-day, or 3-day manufacturing processes described above.

In some embodiments, the time taken from acquiring the lymphocytes to infusing the transduced lymphocytes (vein-to-vein time) is reduced. The vein-to-vein time can be determined by multiple factors, such as the cell transduction and expansion efficiency, as well as the quality and quantity of the starting apheresis material. In some embodiments, taking such factors into consideration, a vein-to-vein time can be projected. In accordance with one embodiment of the present technology, the projected vein-to-vein time is reduced by at least 1 day. In another embodiment, the projected vein-to-vein time is reduced by at least 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days.

In some embodiments, the vein-to-vein time is not longer than 28 days. In some embodiments, the time taken from acquiring the lymphocytes to infusing the transduced lymphocytes is not longer than 27 days. In some embodiments, the vein-to-vein time is not longer than 26 days. In some embodiments, the time taken from acquiring the lymphocytes to infusing the transduced lymphocytes is not longer than 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, or 6 days.

The vein-to-vein time may also depend on the time from the start of LD chemotherapy to infusion. Therefore, in some embodiments, vein-to-vein time can be reduced by reducing the time from the start of LD chemotherapy to infusion. In some embodiments, the time from the start of LD chemotherapy to infusion is not more than 7 days, 6 day, 5 days, 4 days, 3 days, 2 days, or 1 day. In some embodiments, the time from the start of LD chemotherapy to infusion is not more than 5 days.

With such reduced vein-to-vein time, the method can produce transduced lymphocytes such that the patient receiving the infusion has a greater than 55% likelihood of having complete response (CR). In some embodiments, the patient has a greater than 51%, or 52%, 53%, 54%, 56%, 57%, 58%, 59% or 60% likelihood of having complete response.

In some embodiments, the patient has a greater than 45% likelihood of having overall survival (OS) measured at 24 months (following the infusion). In some embodiments, the patient has a greater than 39%, or 40%, 41%, 42%, 43%, 44%, 46%, 47%, 48%, 49%, 50%, 51%, 52% or 53% likelihood of having overall survival (OS) at 24 months (following the infusion).

In some embodiments, the patient has a lower than 30% likelihood of developing prolonged thrombocytopenia. In some embodiments, the patient has a lower than 32%, 31%, 29%, 28%, 27%, 26%, 25%, 24%, 23%, 22%, 21%, 20% or 19% likelihood of developing prolonged thrombocytopenia.

The term “complete response” (CR) refers to a treatment outcome in which the treated patient has evaluable but non-measurable disease, whose tumor and all evidence of disease had disappeared. CR rates can be determined by methods known in the art (e.g., Cheson et al., J Clin Oncol, 2014). In some embodiments, the CR rate can be based on response assessments obtained after the initial administration of the product and prior to any subsequent treatment (e.g., retreatment with the product, subsequent hematopoietic stem cell transplant, and/or other anti-cancer therapy) that may be given for relapse or disease progression.

The term “overall survival” means that at the time of measurement, the patient has not died of any cause.

“Thrombocytopenia” is a condition characterized by abnormally low levels of platelets, also known as thrombocytes, in the blood. A normal human platelet count ranges from 150,000 to 450,000 platelets per microliter of blood. In a patient with thrombocytopenia, the platelet count may be below 50,000 per microliter. “Prolonged thrombocytopenia” refers to a condition in which the patient has thrombocytopenia for at least 30 days after the initial infusion.

In accordance with one embodiment of the present disclosure, the expedited manufacturing process includes an improved transduction/culturing procedure. In some embodiments, the transduction/culturing procedure entails incubating a sample of lymphocytes with a polynucleotide vector to transduce the lymphocytes to produce transduced lymphocytes and culturing the sample that contains the transduced lymphocytes before the lymphocytes are harvested to produce a harvested sample.

In some embodiments, the culturing step is shortened as compared to the conventional process which takes about 4 days. In some embodiment, the culturing step is completed within 96 hours, or within 72 hours, 60 hours, 50 hours, 48 hours, 42 hours, 36 hours, 30 hours, 29 hours, 28 hours, 27 hours, 26 hours, 25 hours, 24 hours, 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, or 4 hours.

In some embodiments, the time for the culturing step is counted from completion of the transduction step (e.g., removal of the cells from the system with immobilized vectors) to harvesting of the cells for storage, transport, or clinical use.

Culturing of transduced lymphocytes can be done in media and conditions known in the art. In some embodiments, the culturing of the transduced lymphocytes may be performed at a temperature and/or in the presence of CO₂. In certain embodiments, the temperature may be about 34° C., about 35° C., about 36° C., about 37° C., about 38° C., or about 39° C. In certain embodiments, the temperature may be about 34-39° C. In certain embodiments, the predetermined temperature may be from about 35-37° C. In certain embodiments, the preferred predetermined temperature may be from about 36-38° C. In certain embodiments, the predetermined temperature may be about 36-37° C. or more preferably about 37° C.

In some embodiments, culturing of the transduced lymphocytes may be performed in the presence of a predetermined level of CO₂. In certain embodiments, the predetermined level of CO₂ may be 1.0-10% CO₂. In certain embodiments, the predetermined level of CO₂ may be about 1.0%, about 2.0%, about 3.0%, about 4.0%, about 5.0%, about 6.0%, about 7.0%, about 8.0%, about 9.0%, or about 10.0% CO₂. In certain embodiments, the predetermined level of CO₂ may be about 4.5-5.5% CO₂. In certain embodiments, the predetermined level of CO₂ may be about 5% CO₂. In certain embodiments, the predetermined level of CO₂ may be about 3.5%, about 4.0%, about 4.5%, about 5.0%, about 5.5%, or about 6.5% CO₂. In some embodiments, the step of expanding the population of transduced T cells may be performed at a predetermined temperature and/or in the presence of a predetermined level of CO₂ in any combination. For example, in one embodiment, the step of expanding the population of transduced T cells may comprise a predetermined temperature of about 36-38° C. and in the presence of a predetermined level of CO₂ of about 4.5-5.5% CO₂.

Any suitable culture medium T cell growth media may be used for culturing the cells in suspension. For example, a T cell growth media may include, but is not limited to, a sterile, low glucose solution that includes a suitable amount of buffer, magnesium, calcium, sodium pyruvate, and sodium bicarbonate. In one embodiment, the culturing media is OpTmizer™ (Life Technologies), but one skilled in the art would understand how to generate similar media.

The incubation (and/or transduction) step can be carried out in a closed system, without limitation. In certain embodiments, the closed system is a closed bag culture system, using any suitable cell culture bags (e.g., Mitenyi Biotec MACS® GMP Cell Differentiation Bags, Origen Biomedical PermaLife™ Cell Culture bags). In some embodiments, the closed system has an inner surface area of at least 500 cm². In some embodiments, the closed system has an inner surface area of at least 1000 cm², 1200 cm², 1400 cm², 1500 cm², 1600 cm², 1800 cm², 2000 cm², 2200 cm², 2500 cm², or 3000 cm². In some embodiments, the closed system has an inner surface area of not greater than 1500 cm², 1600 cm², 1800 cm², 2000 cm², 2200 cm², 2500 cm², or 3000 cm².

In some embodiments, the cell culture bags used in the closed system are coated with a recombinant human fibronectin protein. The recombinant human fibronectin fragment may include three functional domains: a central cell-binding domain, heparin-binding domain II, and a CS1-sequence. The recombinant human fibronectin protein or fragment thereof may be used to increase gene efficiency of viral transduction of immune cells by aiding co-localization of target cells or the vector. In certain embodiments, the recombinant human fibronectin fragment is RetroNectin® (Takara Bio, Japan). In certain embodiments, the cell culture bags may be coated with recombinant human fibronectin fragment at a concentration of about 0.1-60 μg/mL, preferably 0.5-40 μg/mL. In certain embodiments, the cell culture bags may be coated with recombinant human fibronectin fragment at a concentration of about 0.5-20 μg/mL, 20-40 μg/mL, or 40-60 μg/mL. In certain embodiments, the cell culture bags may be coated with about 0.5 μg/mL, 1 μg/mL, about 2 μg/mL, about 3 μg/mL, about 4 μg/mL, about 5 μg/mL, about 6 μg/mL, about 7 μg/mL, about 8 μg/mL, about 9 μg/mL, about 10 μg/mL, about 11 μg/mL, about 12 μg/mL, about 13 μg/mL, about 14 μg/mL, about 15 μg/mL, about 16 μg/mL, about 17 μg/mL, about 18 μg/mL, about 19 μg/mL, or about 20 μg/mL recombinant human fibronectin fragment. In certain embodiments, the cell culture bags may be coated with about 2-5 μg/mL, about 2-10 μg/mL, about 2-20 μg/mL, about 2-25 μg/mL, about 2-30 μg/mL, about 2-35 μg/mL, about 2-40 μg/mL, about 2-50 μg/mL, or about 2-60 μg/mL recombinant human fibronectin fragment. In certain embodiments, the cell culture bags may be coated with at least about 2 μg/mL, at least about 5 μg/mL, at least about 10 μg/mL, at least about 15 μg/mL, at least about 20 μg/mL, at least about 25 μg/mL, at least about 30 μg/mL, at least about 40 μg/mL, at least about 50 μg/mL, or at least about 60 μg/mL recombinant human fibronectin fragment. In certain embodiments, the cell culture bags may be coated with at least about 10 μg/mL recombinant human fibronectin fragment.

In some embodiments, a transduction enhancing agent is introduced into the closed system. Non-limiting examples of such transduction enhancing agents include Vectofusin™ transduction mixtures.

In certain embodiments, the cell culture bags used in the closed bag culture system may be blocked with human albumin serum (HSA). In an alternative embodiment, the cell culture bags are not blocked with HSA.

Once the closed system is coated with the recombinant fibronectin, a solution that includes the vector is added to the closed system so that the vector can be immobilized by the recombinant fibronectin, on the inner surface of the closed system. Such immobilization can improve the transduction efficiency once the cells are added.

In some embodiments, the vectors can be viral vectors, such as lentiviral vectors, as well as retroviral vectors. Several recombinant viruses have been used as viral vectors to deliver genetic material to a cell. Viral vectors that may be used in accordance with the transduction step may be any ecotropic or amphotropic viral vector including, but not limited to, recombinant retroviral vectors, recombinant lentiviral vectors, recombinant adenoviral vectors, and recombinant adeno-associated viral (AAV) vectors. In one embodiment, the viral vector is an MSGV1 gamma retroviral vector. In some embodiments, the vectors are non-viral vectors.

In some embodiments, a total volume of at least 10 mL of the solution that contains the vector is used. In some embodiments, a total volume of at least 100 mL of the solution that contains the vector is used. In some embodiments, a total volume of at least 10 mL, 20 mL, 30 mL, 40 mL, 50 mL, 60 mL, 70 mL, 80 mL, 90 mL, 100 mL, 110 mL, 120 mL, 130 mL, 140 mL, 150 mL, 160 mL, 170 mL, 180 mL, 190 mL, 200 mL, 210 mL, 220 mL, 230 mL, 240 mL, 250 mL, 260 mL, 270 mL, 280 mL, 290 mL, 300 mL, 350 mL, or 400 mL of the solution that contains the vector is used. In some embodiments, a total volume of no more than 150 mL, 160 mL, 170 mL, 180 mL, 190 mL, 200 mL, 210 mL, 220 mL, 230 mL, 240 mL, 250 mL, 260 mL, 270 mL, 280 mL, 290 mL, 300 mL, 350 mL, 400 mL, or 500 mL of the solution that contains the vector is used.

In some embodiments, the vector solution includes at between 1×10³ to 1×10¹² transduction units per milliliter (TU/ml) of the viral vector.

Once the closed system is coated with the recombinant fibronectin and has immobilized the vector, the vector solution can be removed. In some embodiments, the closed system does not include recombinant fibronectin. In some embodiments, the removal of the vector solution is done by gravity or syringe drain, which helps to retain the immobilized vector on the inner surface while removing impurities.

Lymphocyte transduction can be carried in the coated closed system with the immobilized vectors. In some embodiments, the transduction is performed with a sample that contained the lymphocytes. In some embodiments, the sample includes at least 2.5×10⁷ lymphocytes (e.g., T cells). In some embodiments, the sample includes at least 3×10⁷, 4×10⁷, 5×10⁷, 6×10⁷, 7×10⁷, 8×10⁷, 9×10⁷, 1×10⁸, 1.2×10⁸, 1.5×10⁸, 1.8×10⁸, 2×10⁸, 2.2×10⁸, 2.5×10⁸, 2.6×10⁸, 2.7×10⁸, 2.8×10⁸, 2.9×10⁸, 3×10⁸, 3.1×10⁸, 3.2×10⁸, 3.3×10⁸, 3.4×10⁸, 3.5×10⁸, 3.6×10⁸, 3.7×10⁸, 3.8×10⁸, 3.9×10⁸, 4×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, 5×10⁸, 5.1×10⁸, 5.2×10⁸, 5.3×10⁸, 5.4×10⁸, 5.5×10⁸, 5.6×10⁸, 5.7×10⁸, 5.8×10⁸, 5.9×10⁸, 6×10⁸, 6.1×10⁸, 6.2×10⁸, 6.3×10⁸, 6.4×10⁸, 6.5×10⁸, 6.6×10⁸, 6.7×10⁸, 6.8×10⁸, 6.9×10⁸, 7×10⁸, 7.5×10⁸, 8×10⁸, 9×10⁸, or 10×10⁸ lymphocytes (e.g., T cells). In some embodiments, the sample includes no more than 3×10⁸, 3.1×10⁸, 3.2×10⁸, 3.3×10⁸, 3.4×10⁸, 3.5×10⁸, 3.6×10⁸, 3.7×10⁸, 3.8×10⁸, 3.9×10⁸, 4×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, 5×10⁸, 5.1×10⁸, 5.2×10⁸, 5.3×10⁸, 5.4×10⁸, 5.5×10⁸, 5.6×10⁸, 5.7×10⁸, 5.8×10⁸, 5.9×10⁸, 6×10⁸, 6.1×10⁸, 6.2×10⁸, 6.3×10⁸, 6.4×10⁸, 6.5×10⁸, 6.6×10⁸, 6.7×10⁸, 6.8×10⁸, 6.9×10⁸, 7×10⁸, 7.5×10⁸, 8×10⁸, 9×10⁸, or 10×10⁸ lymphocytes (e.g., T cells).

The lymphocytes used in the presently disclosed methods are typically obtained from a donor subject, which may be a cancer patient that is to be treated with a population of cells generated by the methods described herein (i.e., an autologous donor), or may be an individual that donates a lymphocyte sample that, upon generation of the population of cells generated by the methods described herein, will be used to treat a different individual or cancer patient (i.e., an allogeneic donor). The lymphocytes may be obtained from the donor subject by any suitable method used in the art. For example, the lymphocytes may be obtained by any suitable extracorporeal method, venipuncture, or other blood collection method by which a sample of blood and/or lymphocytes is obtained. In one embodiment, the lymphocytes are obtained by apheresis.

Optionally, in some embodiments, the method described herein further includes a step of enriching a population of lymphocytes obtained from the donor subject, prior to the transduction.

Enrichment of lymphocytes may be accomplished by any suitable separation method including, but not limited to, the use of a separation medium (e.g., Ficoll-Paque™, RosetteSep™ HLA Total Lymphocyte enrichment cocktail, Lymphocyte Separation Medium (LSA) (MP Biomedical Cat. No. 0850494X), a non-ionic iodixanol-based medium such as OptiPrep™, or the like), cell size, shape or density separation by filtration or elutriation, immunomagnetic separation (e.g., magnetic-activated cell sorting system, MACS), fluorescent separation (e.g., fluorescence activated cell sorting system, FACS), or bead based column separation.

Optionally, in some embodiments, circulating lymphoma cells are removed from the sample through positive enrichment for CD4⁺/CD8⁺ cells via the use of selection reagents. In some such embodiments, after incubation with the selection reagents, incubated cells, including cells in which the selection reagent has bound are transferred into a system for immunoaffinity-based separation of the cells. In some embodiments, the system for immunoaffinity-based separation is or contains a magnetic separation column.

In some such embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some embodiments includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner. Such separation steps may be based on positive selection, in which the cells having bound the reagents are retained for further use, and/or negative selection, in which the cells having not bound to the antibody or binding partner are retained. In some examples, both fractions are retained for further use.

In some such embodiments, negative selection may be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step may deplete cells expressing multiple markers simultaneously, such as by incubating cells with a plurality of antibodies or binding partners, each specific for a marker targeted for negative selection. Likewise, multiple cell types may simultaneously be positively selected by incubating cells with a plurality of antibodies or binding partners expressed on the various cell types.

For example, in some embodiments, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+T cells, are isolated by positive or negative selection techniques. For example, CD3+, CD28+ T cells may be positively selected using anti-CD3/anti-CD28 conjugated magnetic beads (e.g., DYNABEADS® M-450 CD3/CD28 T Cell Expander). In some embodiments, the population of cells is enriched for T cells with naïve phenotype (CD45RA+CCR7+).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (markerhigh) on the positively or negatively selected cells, respectively.

In particular embodiments, a biological sample, e.g., a sample of PBMCs or other white blood cells, are subjected to selection of CD4+ T cells, where both the negative and positive fractions are retained. In certain embodiments, CD8+ T cells are selected from the negative fraction. In some embodiments, a biological sample is subjected to selection of CD8+ T cells, where both the negative and positive fractions are retained. In certain embodiments, CD4+ T cells are selected from the negative fraction.

In some embodiments, T cells are separated from a PBMC sample by negative selection of markers expressed on non-T cells, such as B cells, monocytes, or other white blood cells, such as CD14. In some embodiments, a CD4+ or CD8+selection step is used to separate CD4+helper and CD8+cytotoxic T cells. Such CD4+ and CD8+populations may be further sorted into sub-populations by positive or negative selection for markers expressed or expressed to a relatively higher degree on one or more naïve, memory, and/or effector T cell subpopulations.

In one example, to enrich for CD4+cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinity magnetic) separation techniques. In some embodiments, the sample or composition of cells to be separated is incubated with small, magnetizable or magnetically responsive material, such as magnetically responsive particles or microparticles, such as paramagnetic beads (e.g., such as Dynabeads™ or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample. In some embodiments, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some embodiments, a combination of positive and negative selection is performed during the same selection step, where the positive and negative fractions are retained and further processed or subject to further separation steps. In some embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies. In some embodiments, the magnetically responsive particles are left attached to the cells that are to be subsequently incubated, cultured and/or engineered; in some embodiments, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotec, Auburn, CA). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they may be eluted and recovered. In certain embodiments, the non-target cells are labelled and depleted from the heterogeneous population of cells.

In some embodiments, the isolation or separation is carried out using a system, device, or apparatus that carries out one or more of the isolation, cell preparation, separation, processing, incubation, culture, and/or formulation steps of the methods. In some embodiments, the system is used to carry out each of these steps in a closed or sterile environment, for example, to minimize error, user handling and/or contamination. In one example, the system is a system as described in International Patent Application, Publication Number WO2009/072003, or US 20110003380 Al, which are each incorporated herein by reference. In some embodiments, the system or apparatus carries out one or more, e.g., ah, of the isolation, processing, engineering, and formulation steps in an integrated or self-contained system, and/or in an automated or programmable fashion. In some embodiments, the system or apparatus includes a computer and/or computer program in communication with the system or apparatus, which allows a user to program, control, assess the outcome of, and/or adjust various embodiments of the processing, isolation, engineering, and formulation steps. In some embodiments, the separation and/or other steps is carried out using CliniMACS system (Miltenyi Biotec), for example, for automated separation of cells on a clinical-scale level in a closed and sterile system. Components may include an integrated microcomputer, magnetic separation unit, peristaltic pump, and various pinch valves. The integrated computer in some embodiments controls ah components of the instrument and directs the system to perform repeated procedures in a standardized sequence. The magnetic separation unit in some embodiments includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some embodiments uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some embodiments is equipped with a cell processing unity that permits automated washing and fractionation of cells by centrifugation. The CliniMACS Prodigy system may also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system may also include an integrated cell cultivation chamber which accomplishes cell culture protocols such as, e.g., cell differentiation and expansion, antigen loading, and long-term cell culture. Input ports may allow for the sterile removal and replenishment of media and cells may be monitored using an integrated microscope.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells may be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, at least 0.5×10⁹ lymphocytes are acquired from the donor, and are optionally enriched and/or subjected to the stimulation. In some embodiments, at least 0.6×10⁹, 0.7×10⁹, 0.8×10⁹, 0.9×10⁹, 1×10⁹, 1.1×10⁹, 1.2×10⁹, 1.3×10⁹, 1.4×10⁹, 1.5×10⁹, 1.6×10⁹, 1.7×10⁹, 1.8×10⁹, 1.9×10⁹, 2×10⁹, 2.5×10⁹, or 3×10⁹ lymphocytes are acquired from the donor, and are optionally enriched and/or subjected to the stimulation. In some embodiments, no more than 1×10⁹, 1.1×10⁹, 1.2×10⁹, 1.3×10⁹, 1.4×10⁹, 1.5×10⁹, 1.6×10⁹, 1.7×10⁹, 1.8×10⁹, 1.9×10⁹, 2×10⁹, 2.5×10⁹, or 3×10⁹ lymphocytes are acquired from the donor, and are optionally enriched and/or subjected to the stimulation.

Also optionally, the methods described herein further includes a step of stimulating the lymphocytes with one or more lymphocyte stimulating agents. In some embodiments, the stimulation is performed prior to the transduction step. In some embodiments, the stimulation is performed after the transduction step.

Any combination of one or more suitable lymphocyte stimulating agents may be used to stimulate (activate) the lymphocytes. Non-limiting examples include an antibody or functional fragment thereof which targets a T-cell stimulatory or co-stimulatory molecule (e.g., anti-CD2 antibody, anti-CD3 antibody, anti-CD28 antibody, or functional fragments thereof) a T cell cytokine (e.g., any isolated, wildtype, or recombinant cytokines such as: interleukin 1 (IL-1), interleukin 2, (IL-2), interleukin 4 (IL-4), interleukin 5 (IL-5), interleukin 7 (IL-7), interleukin 15 (IL-15), tumor necrosis factor α (TNFα)), or any other suitable mitogen (e.g., tetradecanoyl phorbol acetate (TPA), phytohaemagglutinin (PHA), concanavalin A (conA), lipopolysaccharide (LPS), pokeweed mitogen (PWM)) or natural ligand to a T-cell stimulatory or co-stimulatory molecule. In some embodiments, the stimulating agent is an anti-CD3 antibody and/or an anti-CD28 antibody.

In some embodiments, the step of stimulating lymphocytes as described herein may entail stimulating the lymphocytes with one or more stimulating agents at a predetermined temperature, for a predetermined amount of time, and/or in the presence of a predetermined level of CO₂. In certain embodiments, the predetermined temperature for stimulation may be about 34° C., about 35° C., about 36° C., about 37° C., about 38° C., or about 39° C. In certain embodiments, the predetermined temperature for stimulation may be about 34-39° C. In certain embodiments, the step of stimulating the lymphocytes comprises stimulating the lymphocytes with one or more stimulating agents for a predetermined time. In certain embodiments, the predetermined time for stimulation may be about 24-72 hours. In certain embodiments, the predetermined time for stimulation may be about 24-36 hours. In certain embodiments, the step of stimulating the lymphocytes may comprise stimulating the lymphocytes with one or more stimulating agents in the presence of a predetermined level of CO₂. In certain embodiments, the predetermined level of CO₂ for stimulation may be about 1.0-10% CO₂. In certain embodiments, the predetermined level of CO₂ for stimulation may be about 1.0%, about 2.0%, about 3.0%, about 4.0%, about 5.0%, about 6.0%, about 7.0%, about 8.0%, about 9.0%, or about 10.0% CO₂.

In some embodiments, an anti-CD3 antibody (or functional fragment thereof), an anti-CD28 antibody (or functional fragment thereof), or a combination of anti-CD3 and anti-CD28 antibodies may be used in accordance with the step of stimulating the population of lymphocytes. Any soluble or immobilized anti-CD3 and/or anti-CD28 antibody or functional fragment thereof may be used (e.g., clone OKT3 (anti-CD3), clone 145-2C11 (anti-CD3), clone UCHT1 (anti-CD3), clone L293 (anti-CD28), clone 15E8 (anti-CD28)). In some aspects, the antibodies may be purchased commercially from vendors known in the art including, but not limited to, Miltenyi Biotec, BD Biosciences (e.g., MACS GMP CD3 pure 1 mg/mL, Part No. 170-076-116), and eBioscience, Inc. Further, one skilled in the art would understand how to produce an anti-CD3 and/or anti-CD28 antibody by standard methods. Any antibody used in the methods described herein should be produced under Good Manufacturing Practices (GMP) to conform to relevant agency guidelines for biologic products.

In certain embodiments, the T cell stimulating agent may include an anti-CD3 or anti-CD28 antibody at a concentration of from about 20 ng/mL-100 ng/mL. In certain embodiments, the concentration of anti-CD3 or anti-CD28 antibody may be about 20 ng/mL, about 30 ng/mL, about 40 ng/mL, about 50 ng/mL, about 60 ng/mL, about 70 ng/mL, about 80 ng/mL, about 90 ng/mL, or about 100 ng/mL.

The lymphocytes prepared, as demonstrated in the 7-day, 5-day, and 3-day processes described above, include higher ratios of juvenile ones (e.g., naïve T cells). Accordingly, one embodiment of the present disclosure provides a population of lymphocytes, prepared by the instant methods, that include CD4+ and CD8+ T cells.

In some embodiments, at least 20% of the CD4+ T cells are naïve T cells. In some embodiments, at least 25%, 30%, 35%, 40%, 45%, 50%, 55% or 60% of the CD4+ T cells are naïve T cells.

In some embodiments, no more than 25% of the CD4+ T cells are effector memory T cells. In some embodiments, no more than 20%, 19%, 18%, 17%, 16%, 15%, 14%, 13%, 12%, 11%, 10%, 9%, 8%, 7%, 6% or 5% of the CD4+ T cells are effector memory T cells.

In some embodiments, no more than 44% of the CD4+ T cells are central memory T cells.

In some embodiments, no more than 43%, 42%, 41%, or 40% of the CD4+ T cells are central memory T cells.

In some embodiments, no more than 1.5% of the CD4+ T cells are effector T cells. In some embodiments, no more than 1.4%, 1.3%, 1.2%, 1.1%, 1%, 0.9%, 0.8%, 0.7%, 0.6% or 0.5% of the CD4+ T cells are effector T cells.

In some embodiments, at least 5% of the CD8+ T cells are naïve T cells. In some embodiments, at least 10%, 15%, 20%, 25%, 30% or 35% of the CD8+ T cells are naïve T cells.

In some embodiments, no more than 30% of the CD8+ T cells are effector memory T cells.

In some embodiments, no more than 28%, 27%, 25%, 22%, 20%, 19%, 18%, 17%, 16%, 15%, 14%, 13%, 12%, 11%, or 10% of the CD8+ T cells are effector memory T cells.

In some embodiments, no more than 60% of the CD8+ T cells are central memory T cells. In some embodiments, no more than 58%, 56%, 55%, 54%, 52% or 50% of the CD8+ T cells are central memory T cells.

Each type of T cells can be characterized with cell surface markers, as well known in the art. For instance, naïve T cells can be characterized as CCR7+, CD45RO−, and CD95−. Additional markers for naïve T cell include CD45RA+, CD62L+, CD27+, CD28+, CD127+, CD132+, CD25−, CD44−, and HLA-DR−.

Surface markers to stem memory T cells (Tscm) include, without limitation, CD45RO−, CCR7+, CD45RA+, CD62L+(L-selectin), CD27+, CD28+, IL-7Ra+, CD95+, IL-2RP+, CXCR3+, and LFA−.

Surface markers for effector memory T cells (Tem) include, without limitation, CCR7−, CD45RO+ and CD95+. Additional marker for effector memory T cells is IL-2RP+. For central memory T cells (Tcm), suitable markers include CD45RO+, CD95+, IL-2RP+, CCR7+ and CD62L+. For effector T cells (Teff), suitable markers include CD45RA+, CD95+, IL-2RP+, CCR7− and CD62L−, without limitation.

The harvested lymphocytes preferably include a good proportion that is CD3+ T cells. In some embodiments, at least 25%, 35%, 45%, 50%, 55%, 60%, 65%, 70%, 75%, 80%, 85% or 90% of the harvested lymphocytes are CD3+ T cells.

The harvested lymphocytes preferably include a good proportion that has been transduced. In some embodiments, at least 15%, 20%, 25%, 30%, 35%, 40%, 45%, or 50% of the harvested lymphocytes are transduced with the vector. In some embodiments, each transduced lymphocyte includes at least a copy of the vector (or the included coding sequence) integrated to the host genome. In some embodiments, each transduced lymphocyte includes at least 2, 3, 4, 5, 6, 7, 8, 9 or 10 copies of the vector integrated to the host genome.

In some embodiments, the vector includes a transgene that encodes a polypeptide. The polypeptide, without limitation, may be a CAR or TCR. In some embodiments, the CAR or TCR includes an antigen binding molecule. The antigen binding molecule, in some embodiments, has binding specificity to an antigenic moiety. In some embodiments, the antigen binding molecule has binding specificity to one or more antigenic moieties (e.g., 1, 2, 3, or 4 antigenic moieties). In some embodiments, the antigen binding molecule has binding specificity to two different antigenic moieties.

In some aspects, the antigenic moiety is associated with a cancer or a cancer cell. Such antigenic moieties may include, but are not limited to, 707-AP (707 alanine proline), AFP (alpha (a)-fetoprotein), ART-4 (adenocarcinoma antigen recognized by T4 cells), BAGE (B antigen; b-catenin/m, b-catenin/mutated), BCMA (B cell maturation antigen), Bcr-abl (breakpoint cluster region-Abelson), CAIX (carbonic anhydrase IX), CD19 (cluster of differentiation 19), CD20 (cluster of differentiation 20), CD22 (cluster of differentiation 22), CD30 (cluster of differentiation 30), CD33 (cluster of differentiation 33), CD44v7/8 (cluster of differentiation 44, exons 7/8), CAMEL (CTL-recognized antigen on melanoma), CAP-1 (carcinoembryonic antigen peptide—1), CASP-8 (caspase-8), CDC27m (cell-division cycle 27 mutated), CDK4/m (cycline-dependent kinase 4 mutated), CEA (carcinoembryonic antigen), CT (cancer/testis (antigen)), Cyp-B (cyclophilin B), DAM (differentiation antigen melanoma), EGFR (epidermal growth factor receptor), EGFRvIII (epidermal growth factor receptor, variant III), EGP-2 (epithelial glycoprotein 2), EGP-40 (epithelial glycoprotein 40), Erbb2, 3, 4 (erythroblastic leukemia viral oncogene homolog-2, -3, 4), ELF2M (elongation factor 2 mutated), ETV6-AML1 (Ets variant gene 6/acute myeloid leukemia 1 gene ETS), FBP (folate binding protein), fAchR (Fetal acetylcholine receptor), G250 (glycoprotein 250), GAGE (G antigen), GD2 (disialoganglioside 2), GD3 (disialoganglioside 3), GnT-V (N-acetylglucosaminyltransferase V), Gp100 (glycoprotein 100 kD), HAGE (helicose antigen), HER-2/neu (human epidermal receptor-2/neurological; also known as EGFR2), HLA-A (human leukocyte antigen-A) HPV (human papilloma virus), HSP70-2M (heat shock protein 70-2 mutated), HST-2 (human signet ring tumor 2), hTERT or hTRT (human telomerase reverse transcriptase), iCE (intestinal carboxyl esterase), IL-13R-a2 (Interleukin-13 receptor subunit alpha-2), KIAA0205, KDR (kinase insert domain receptor), κ-light chain, LAGE (L antigen), LDLR/FUT (low density lipid receptor/GDP-L-fucose: b-D-galactosidase 2-a-Lfucosyltransferase), LeY (Lewis-Y antibody), LiCAM (L1 cell adhesion molecule), MAGE (melanoma antigen), MAGE-A1 (Melanoma-associated antigen 1), mesothelin, Murine CMV infected cells, MART-1/Melan-A (melanoma antigen recognized by T cells-1/Melanoma antigen A), MC1R (melanocortin 1 receptor), Myosin/m (myosin mutated), MUC1 (mucin 1), MUM-1, -2, -3 (melanoma ubiquitous mutated 1, 2, 3), NA88-A (NA cDNA clone of patient M88), NKG2D (Natural killer group 2, member D) ligands, NY-BR-1 (New York breast differentiation antigen 1), NY-ESO-1 (New York esophageal squamous cell carcinoma-1), oncofetal antigen (h5T4), P15 (protein 15), p190 minor bcr-abl (protein of 190KD bcr-abl), Pml/RARa (promyelocytic leukaemia/retinoic acid receptor a), PRAME (preferentially expressed antigen of melanoma), PSA (prostate-specific antigen), PSCA (Prostate stem cell antigen), PSMA (prostate-specific membrane antigen), RAGE (renal antigen), RU1 or RU2 (renal ubiquitous 1 or 2), SAGE (sarcoma antigen), SART-1 or SART-3 (squamous antigen rejecting tumor 1 or 3), SSX1, -2, -3, 4 (synovial sarcoma X1, -2, -3, -4), TAA (tumor-associated antigen), TAG-72 (Tumor-associated glycoprotein 72), TEL/AML1 (translocation Ets-family leukemia/acute myeloid leukemia 1), TPI/m (triosephosphate isomerase mutated), TRP-1 (tyrosinase related protein 1, or gp75), TRP-2 (tyrosinase related protein 2), TRP-2/INT2 (TRP-2/intron 2), VEGF-R2 (vascular endothelial growth factor receptor 2), or WT1 (Wilms' tumor gene), or a combination thereof.

Additional examples of cancer cell-associated antigens include 2B4 (CD244), 4-1BB, 5T4, A33 antigen, adenocarcinoma antigen, adrenoceptor beta 3 (ADRB3), A kinase anchor protein 4 (AKAP-4), alpha-fetoprotein (AFP), anaplastic lymphoma kinase (ALK), Androgen receptor, B7H3 (CD276), 02-integrins, BAFF, B-lymphoma cell, B cell maturation antigen (BCMA), bcr-abl (oncogene fusion protein consisting of breakpoint cluster region (BCR) and Abelson murine leukemia viral oncogene homolog 1 (Abl), BhCG, bone marrow stromal cell antigen 2 (BST2), CCCTC-Binding Factor (Zinc Finger Protein)-Like (BORIS or Brother of the Regulator of Imprinted Sites), BST2, C242 antigen, 9-0-acetyl-CA19-9 marker, CA-125, CAEX, calreticulin, carbonic anhydrase 9 (CAIX), C-MET, CCR4, CCR5, CCR8, CD2, CD3, CD4, CD5, CD8, CD7, CD10, CD16, CD19, CD20, CD22, CD23 (IgE receptor), CD24, CD25, CD27, CD28, CD30 (TNFRSF8), CD33, CD34, CD38, CD40, CD40L, CD41, CD44, CD44V6, CD49f, CD51, CD52, CD56, CD63, CD70, CD72, CD74, CD79a, CD79b, CD80, CD84, CD96, CD97, CD100, CD123, CD125, CD133, CD137, CD138, CD150, CD152 (CTLA-4), CD160, CD171, CD179a, CD200, CD221, CD229, CD244, CD272 (BTLA), CD274 (PDL-1, B7H1), CD279 (PD-1), CD352, CD358, CD300 molecule-like family member f (CD300LF), Carcinoembryonic antigen (CEA), claudin 6 (CLDN6), C-type lectin-like molecule-1 (CLL-1 or CLECL1), C-type lectin domain family 12 member A (CLEC12A), a cytomegalovirus (CMV) infected cell antigen, CNT0888, CRTAM (CD355), CS-1 (also referred to as CD2 subset 1, CRACC, CD319, and 19A24), CTLA-4, Cyclin B 1, chromosome X open reading frame 61 (CXORF61), Cytochrome P450 1B 1 (CYP1B1), DNAM-1 (CD226), desmoglein 4, DR3, DR5, E-cadherin neoepitope, epidermal growth factor receptor (EGFR), EGF1R, epidermal growth factor receptor variant III (EGFRvIII), epithelial glycoprotein-2 (EGP-2), epithelial glycoprotein-40 (EGP-40), EGF-like module-containing mucin-like hormone receptor-like 2 (EMR2), elongation factor 2 mutated (ELF2M), endosialin, Epithelial cell adhesion molecule (EPCAM), ephrin type-A receptor 2 (EphA2), Ephrin B2, receptor tyrosine-protein kinases erb-B2,3,4 (erb-B2,3,4), ERBB, ERBB2 (Her2/neu), ERG (transmembrane protease, serine 2 (TMPRSS2) ETS fusion gene), ETA, ETS translocation-variant gene 6, located on chromosome 12p (ETV6-AML), Fc fragment of IgA receptor (FCAR or CD89), fibroblast activation protein alpha (FAP), FBP, Fc receptor-like 5 (FCRL5), fetal acetylcholine receptor (AChR), fibronectin extra domain-B, Fms-Like Tyrosine Kinase 3 (FLT3), folate-binding protein (FBP), folate receptor 1, folate receptor α, Folate receptor β, Fos-related antigen 1, Fucosyl, Fucosyl GM1; GM2, ganglioside G2 (GD2), ganglioside GD3 (aNeu5Ac(2-8)aNeu5Ac(2-3)bDGalp(1-4)bDGlcp(1-1)Cer), o-acetyl-GD2 ganglioside (OAcGD2), GITR (TNFRSF 18), GM1, ganglioside GM3 (aNeu5Ac(2-3)bDGalp(1-4)bDGlcp(1-1)Cer), GP 100, hexasaccharide portion of globoH glycoceramide (GloboH), glycoprotein 75, Glypican-3 (GPC3), glycoprotein 100 (gplOO), GPNMB, G protein-coupled receptor 20 (GPR20), G protein-coupled receptor class C group 5, member D (GPRC5D), Hepatitis A virus cellular receptor 1 (HAVCRi), human Epidermal Growth Factor Receptor 2 (HER-2), HER2/neu, HER3, HER4, HGF, high molecular weight-melanoma-associated antigen (HMWMAA), human papilloma virus E6 (HPV E6), human papilloma virus E7 (HPV E7), heat shock protein 70-2 mutated (mut hsp70-2), human scatter factor receptor kinase, human Telomerase reverse transcriptase (hTERT), HVEM, ICOS, insulin-like growth factor receptor 1 (IGF-1 receptor), IGF-I, IgG1, immunoglobulin lambda-like polypeptide 1 (IGLL1), IL-6, Interleukin 11 receptor alpha (IL-11Ra), IL-13, Interleukin-13 receptor subunit alpha-2 (IL-13Ra2 or CD213A2), insulin-like growth factor I receptor (IGF1-R), integrin a5p 1, integrin avP3, intestinal carboxyl esterase, κ-light chain, KCS1, kinase insert domain receptor (KDR), KIR, KIR2DL1, KIR2DL2, KIR2DL3, KIR3DL2, KIR-L, KG2D ligands, KIT (CD117), KLRGI, LAGE-la, LAG3, lymphocyte-specific protein tyrosine kinase (LCK), Leukocyte immunoglobulin-like receptor subfamily A member 2 (LILRA2), legumain, Leukocyte-associated immunoglobulin-like receptor 1 (LAIR1), Lewis(Y) antigen, LeY, LG, LI cell adhesion molecule (LI-CAM), LIGHT, LMP2, lymphocyte antigen 6 complex, LTBR, locus K 9 (LY6K), Ly-6, lymphocyte antigen 75 (LY75), melanoma cancer testis antigen-1 (MAD-CT-1); melanoma cancer testis antigen-2 (MAD-CT-2), MAGE, Melanoma-associated antigen 1 (MAGE-A1), MAGE-A3 melanoma antigen recognized by T cells 1 (MelanA or MARTI), MelanA/MARTl, Mesothelin, MAGE A3, melanoma inhibitor of apoptosis (ML-IAP), melanoma-specific chondroitin-sulfate proteoglycan (MCSCP), MORAb-009, MS4A1, Mucin 1 (MUCl), MUC2, MUC3, MUC4, MUC5AC, MUC5b, MUC7, MUC16, mucin CanAg, Mullerian inhibitory substance (MIS) receptor type II, v-myc avian myelocytomatosis viral oncogene neuroblastoma derived homolog (MYCN), N-glycolylneuraminic acid, N-Acetyl glucosaminyl-transferase V (NA17), neural cell adhesion molecule (NCAM), NKG2A, NKG2C, NKG2D, NKG2E ligands, NKR-P IA, NPC-1C, NTB-A, mammary gland differentiation antigen (NY-BR-1), NY-ESO-1, oncofetal antigen (h5T4), Olfactory receptor 51E2 (OR51E2), OX40, plasma cell antigen, poly SA, proacrosin binding protein sp32 (OY-TES 1), p53, p53 mutant, pannexin 3 (PANX3), prostatic acid phosphatase (PAP), paired box protein Pax-3 (PAX3), Paired box protein Pax-5 (PAX5), prostate carcinoma tumor antigen-1 (PCTA-1 or Galectin 8), PD-1H, Platelet-derived growth factor receptor alpha (PDGFR-alpha), PDGFR-beta, PDL192, PEN-5, phosphatidylserine, placenta-specific 1 (PLAC1), Polysialic acid, Prostase, prostatic carcinoma cells, prostein, Protease Serine 21 (Testisin or PRSS21), Proteinase3 (PR1), prostate stem cell antigen (PSCA), prostate-specific membrane antigen (PSMA), Proteasome (Prosome, Macropain) Subunit, Beta Type, Receptor for Advanced Glycation Endproducts (RAGE-1), RANKL, Ras mutant, Ras Homolog Family Member C (RhoC), RON, Receptor tyrosine kinase-like orphan receptor 1 (ROR1), renal ubiquitous 1 (RUl), renal ubiquitous 2 (RU2), sarcoma translocation breakpoints, Squamous Cell Carcinoma Antigen Recognized By T Cells 3 (SART3), SAS, SDC1, SLAMF7, sialyl Lewis adhesion molecule (sLe), Siglec-3, Siglec-7, Siglec-9, sonic hedgehog (SHH), sperm protein 17 (SPA17), Stage-specific embryonic antigen-4 (SSEA-4), STEAP, sTn antigen, synovial sarcoma, X breakpoint 2 (SSX2), Survivin, Tumor-associated glycoprotein 72 (TAG72), TCR5y, TCRa, TCRB, TCR Gamma Alternate Reading Frame Protein (TARP), telomerase, TIGIT TNF-α precursor, tumor endothelial marker 1 (TEM1/CD248), tumor endothelial marker 7-related (TEM7R), tenascin C, TGF beta 2, TGF-β, transglutaminase 5 (TGS5), angiopoietin-binding cell surface receptor 2 (Tie 2), TIM1, TIM2, TIM3, Tn Ag, TRAIL-R1, TRAIL-R2, Tyrosinase-related protein 2 (TRP-2), thyroid stimulating hormone receptor (TSHR), tumor antigen CTAA16.88, Tyrosinase, ROR1, TAG-72, uroplakin 2 (UPK2), VEGF-A, VEGFR-1, vascular endothelial growth factor receptor 2 (VEGFR2), and vimentin, Wilms tumor protein (WT1), or X Antigen Family, Member 1A (XAGE1), or a combination thereof.

In other embodiments, the antigenic moiety is associated with virally infected cells (i.e., a viral antigenic moiety). Such antigenic moieties may include, but are not limited to, an Epstein-Barr virus (EBV) antigen (e.g., EBNA-1, EBNA-2, EBNA-3, LMP-1, LMP-2), a hepatitis A virus antigen (e.g., VP1, VP2, VP3), a hepatitis B virus antigen (e.g., HBsAg, HBcAg, HBeAg), a hepatitis C viral antigen (e.g., envelope glycoproteins E1 and E2), a herpes simplex virus type 1, type 2, or type 8 (HSV1, HSV2, or HSV8) viral antigen (e.g., glycoproteins gB, gC, gC, gE, gG, gH, gI, gJ, gK, gL. gM, UL20, UL32, US43, UL45, UL49A), a cytomegalovirus (CMV) viral antigen (e.g., glycoproteins gB, gC, gC, gE, gG, gH, gI, gJ, gK, gL. gM or other envelope proteins), a human immunodeficiency virus (HIV) viral antigen (glycoproteins gp120, gp41, or p24), an influenza viral antigen (e.g., hemagglutinin (HA) or neuraminidase (NA)), a measles or mumps viral antigen, a human papillomavirus (HPV) viral antigen (e.g., L1, L2), a parainfluenza virus viral antigen, a rubella virus viral antigen, a respiratory syncytial virus (RSV) viral antigen, or a varicella-zostser virus viral antigen, or a combination thereof. In such embodiments, the cell surface receptor may be any TCR, or any CAR which recognizes any of the aforementioned viral antigens on a target virally infected cell.

In other embodiments, the antigenic moiety is associated with cells having an immune or inflammatory dysfunction. Such antigenic moieties may include, but are not limited to, myelin basic protein (MBP) myelin proteolipid protein (PLP), myelin oligodendrocyte glycoprotein (MOG), carcinoembryonic antigen (CEA), pro-insulin, glutamine decarboxylase (GAD65, GAD67), heat shock proteins (HSPs), or any other tissue specific antigen that is involved in or associated with a pathogenic autoimmune process, or a combination thereof.

In some embodiments, the TCR has specificity to an antigenic moiety on cancer cells. Non-limiting examples of TCR include an anti-707-AP TCR, anti-AFP TCR, anti-ART-4 TCR, anti-BAGE TCR, anti-Bcr-abl TCR, anti-CAMEL TCR, anti-CAP-1 TCR, anti-CASP-8 TCR, anti-CDC27m TCR, anti-CDK4/m TCR, anti-CEA TCR, anti-CT TCR, anti-Cyp-B TCR, anti-DAM TCR, anti-TCR, anti-EGFRvIII TCR, anti-ELF2M TCR, anti-ETV6-AML1 TCR, anti-G250 TCR, GAGE TCR, anti-GnT-V TCR, anti-Gp100 TCR, anti-HAGE TCR, anti-HER-2/neu TCR, anti-HLA-A TCR, anti-HPV TCR, anti-HSP70-2M TCR, anti-HST-2 TCR, anti-hTERT TCR or anti-hTRT TCR, anti-iCE TCR, anti-KIAA0205, anti-LAGE (L antigen), anti-LDLR/FUT TCR, anti-MAGE TCR, anti-MART-1/Melan-A TCR, anti-MC1R TCR, anti-Myosin/m TCR, anti-MUC1 TCR, anti-MUM-1, -2, -3 TCR, anti-NA88-A TCR, anti-NY-ESO-1 TCR, anti-P15 TCR, anti-p190 minor bcr-abl TCR, anti-Pml/RARa TCR, anti-PRAME TCR, anti-PSA TCR, anti-PSMA TCR, anti-RAGE TCR, anti-RU1 TCR or anti-RU2 TCR, anti-SAGE TCR, anti-SART-1 TCR or anti-SART-3 TCR, anti-SSX1, -2, -3,4 TCR, anti-TEL/AML1 TCR, anti-TPI/m TCR, anti-TRP-1 TCR, anti-TRP-2 TCR, anti-TRP-2/INT2 TCR, or anti-WT1 TCR, or a combination thereof.

In some embodiments, a TCR can bind to a first antigenic moiety and a second antigenic moiety. In some embodiments, a first TCR binds to a first antigenic moiety and a second TCR binds to a second antigenic moiety.

The present disclosure can involve the use of a dual-targeted antigen binding system. Dual-targeted antigen binding systems can comprise bispecific CARs or TCRs and/or bicistronic CARs or TCRs. Bispecific and bicistronic CARs can comprise two binding motifs (in a single CAR molecule or in two CAR molecules, respectively). In some embodiments, the vector encodes bicistronic and/or bispecific CARs (e.g., bicistronic and/or bispecific CARs that bind CD20 and CD19). Exemplary bispecific and bicistronic CARs are described in WO2020/123691, incorporated herein by reference.

In some embodiments, a CAR includes a first scFv that binds CD19 and a second scFv that binds CD20. In some embodiments, a first CAR includes a first scFV that binds to CD19 and a second CAR includes a second scFV that binds to CD20. Example CD19- or CD20-binding sequences are provided in Table 1.

TABLE 1
Example Antigen-Binding Sequences

| Name | Sequence |
|---|---|
| Anti-CD20 v01 | SEQ ID NO: 1 |
| VH/VL | QVQLQQWGAGLLKPSETLSLICAVYGGSFSGYYWSWIRQPPGKGLEWIGEIDH |
| | SGSTNYNPSLKSRVTISVDTSKNQFSLKLSSVTAADTAVYYCARGGGSWYSNW |
| | FDPWGQGTMVTVSS |
| | SEQ ID NO: 2 |
| | DIQMTQSPSTLSASVGDRVTITCRASQSISSWLAWYQQKPGKAPKLLIYDASS |
| | LESGVPSRFSGSGSGTEFTLTISSLQPDDFATYYCQQDRSLPPTFGGGTKVEI |
| | K |
| Anti-CD20 v02 | SEQ ID NO: 3 |
| VH/VL | QVQLQQWGAGLLKPSETLSLTCAVYGGSFSGIHWNWIRQPPGKGLEWIGDIDT |
| | SGSTNYNPSLKSRVTISVDTSKNQFSLKLSSVTAADTAVYYCARLGQESATYL |
| | GMDVWGQGTTVTVSS |
| | SEQ ID NO: 4 |
| | DIVMTQSPDSLAVSLGERATINCKSSQSVLYSSNNKNYLAWYQQKPGQPPKLL |
| | IYWASTRESGVPDRFSGSGSGTDFTLTISSLQAEDVAVYYCQQLYTYPFTFGG |
| | GTKVEIK |
| Anti-CD20 v03 | SEQ ID NO: 5 |
| VH/VL | QLQLQESGPGLVKPSETLSLTCTVSGGSISSSSYYWGWIRQPPGKGLEWIGSI |
| | YYSGSTYYNPSLKSRVTISVDTSKNQFSLKLSSVTAADTAVYYCARETDYSSG |
| | MGYGMDVWGQGTTVTVSS |
| | SEQ ID NO: 6 |
| | DIQMTQSPSSLSASVGDRVTITCRASQSINSYLNWYQQKPGKAPKLLIYAASS |
| | LQSGVPSRFSGSGSGTDFTLTISSLQPEDFATYYCQQSLADPFTFGGGTKVEI |
| | K |
| Anti-CD20 v04 | SEQ ID NO: 7 |
| VH/VL | QVQLVQSGAEVKKPGASVKVSCKASGYTFKEYGISWVRQAPGQGLEWMGWISA |
| | YSGHTYYAQKLQGRVTMTTDISTSTAYMELRSLRSDDTAVYYCARGPHYDDWS |
| | GFIIWFDPWGQGTLVTVSS |
| | SEQ ID NO: 8 |
| | DIQMTQSPSSLSASVGDRVTITCRASQSISSYLNWYQQKPGKAPKLLIYAASS |
| | LQSGVPSRFSGSGSGTDFTLTISSLQPEDFATYYCQQSYRFPPTFGQGTKVEI |
| | K |
| Anti-CD20 v05 | SEQ ID NO: 9 |
| VH/VL | QVQLQESGPGLVKPSETLSLTCTVSGGSISSPDHYWGWIRQPPGKGLEWIGSI |
| | YASGSTFYNPSLKSRVTISVDTSKNQFSLKLSSVTAADTAVYYCARETDYSSG |
| | MGYGMDVWGQGTTVTVSS |
| | SEQ ID NO: 10 |
| | DIQMTQSPSSLSASVGDRVTITCRASQSINSYLNWYQQKPGKAPKLLIYAASS |
| | LQSGVPSRFSGSGSGTDFTLTISSLQPEDFATYYCQQSLADPFTFGGGTKVEI |
| | K |
| Anti-CD20 v06 | SEQ ID NO: 11 |
| VH/VL | QITLKESGPTLVKPTQTLTLTCTFSGFSLDTEGVGVGWIRQPPGKALEWLALI |
| | YFNDQKRYSPSLKSRLTITKDTSKNQVVLTMTNMDPVDTAVYYCARDTGYSRW |
| | YYGMDVWGQGTTVTVSS |
| | SEQ ID NO: 12 |
| | DIQMTQSPSSVSASVGDRVTITCRASQGISSWLAWYQQKPGKAPKLLIYAASS |
| | LQSGVPSRFSGSGSGTDFTLTISSLQPEDFATYYCQQAYAYPITFGGGTKVEI |
| | K |
| Anti-CD20 v07 | SEQ ID NO: 13 |
| VH/VL | QVQLQQWGAGLLKPSETLSLTCAVYGGSFEKYYWSWIRQPPGKGLEWIGEIYH |
| | SGLTNYNPSLKSRVTISVDTSKNQFSLKLSSVTAADTAVYYCARVRYDSSDSY |
| | YYSYDYGMDVWGQGTTVTVSS |
| | SEQ ID NO: 14 |
| | DIVLTQSPDSLAVSLGERATINCKSSQSVLYSSNNKNYLAWYQQKPGQPPKLL |
| | IYWASSRESGVPDRFSGSGSGTDFTLTISSLQAEDVAVYYCQQSYSFPWTFGG |
| | GTKVEIK |
| Anti-CD20 v08 | SEQ ID NO: 15 |
| VH/VL | QVQLQQWGAGLLKPSETLSLTCAVYGGSFSRYVWSWIRQPPGKGLEWIGEIDS |
| | SGKTNYNPSLKSRVTISVDTSKNQFSLKLSSVTAADTAVYYCARVRYDSSDSY |
| | YYSYDYGMDVWGQGTTVTVSS |
| | SEQ ID NO: 16 |
| | DIVLTQSPDSLAVSLGERATINCKSSQSVLYSSNNKNYLAWYQQKPGQPPKLL |
| | IYWASSRESGVPDRFSGSGSGTDFTLTISSLQAEDVAVYYCQQSYSFPWTFGG |
| | GTKVEIK |
| Anti-CD20 v09 | SEQ ID NO: 17 |
| VH/VL | QVQLQQWGAGLLKPSETLSLTCAVYGGSFSGYAWSWIRQPPGKGLEWIGEIDH |
| | RGFTNYNPSLKSRVTISVDTSKNQFSLKLSSVTAADTAVYYCARVRYDSSDSY |
| | YYSYDYGMDVWGQGTTVTVSS |
| | SEQ ID NO: 18 |
| | DIVLTQSPDSLAVSLGERATINCKSSQSVLYSSNNKNYLAWYQQKPGQPPKLL |
| | IYWASSRESGVPDRFSGSGSGTDFTLTISSLQAEDVAVYYCQQSYSFPWTFGG |
| | GTKVEIK |
| Anti-CD20 v10 | SEQ ID NO: 19 |
| VH/VL | QVQLQQWGAGLLKPSETLSLTCAVYGGSFQKYYWSWIRQPPGKGLEWIGEIDT |
| | SGFTNYNPSLKSRVTISVDTSKNQFSLKLSSVTAADTAVYYCARVGRYSYGYY |
| | ITAFDIWGQGTTVTVSS |
| | SEQ ID NO: 20 |
| | DIVMTQSPDSLAVSLGERATINCKSSQSVLYSSNNKNYLAWYQQKPGQPPKLL |
| | IYWASTRESGVPDRFSGSGSGTDFTLTISSLQAEDVAVYYCQQHYSFPFTFGG |
| | GTKVEIK |
| Anti-CD19 | SEQ ID NO: 21 |
| VH/VL v01 | EVKLQESGPGLVAPSQSLSVTCTVSGVSLPDYGVSWIRQPPRKGLEWLGVIWG |
| | SETTYYNSALKSRLTIIKDNSKSQVFLKMNSLQTDDTAIYYCAKHYYYGGSYA |
| | MDYWGQGTSVTVSS |
| | SEQ ID NO: 22 |
| | DIQMTQTTSSLSASLGDRVTISCRASQDISKYLNWYQQKPDGTVKLLIYHTSR |
| | LHSGVPSRFSGSGSGTDYSLTISNLEQEDIATYFCQQGNTLPYTFGGGTKLEI |
| | T |
| Anti-CD19 | SEQ ID NO: 23 |
| VH/VL v02 | EVQLVESGGGLVQPGRSLRLSCTASGVSLPDYGVSWIRQPPGKGLEWIGVIWG |
| | SETTYYNSALKSRFTISRDNSKNTLYLQMNSLRAEDTAVYYCAKHYYYGGSYA |
| | MDYWGQGTLVTVSS |
| | SEQ ID NO: 24 |
| | DIQMTQSPSSLSASVGDRVTITCRASQDISKYLNWYQQKPDQAPKLLIKHTSR |
| | LHSGVPSRFSGSGSGTDYTLTISSLQPEDFATYYCQQGNTLPYTFGQGTKLEI |
| | K |
| Anti-CD19 scFv | SEQ ID NO: 25 |
| | DIQMTQTTSSLSASLGDRVTISCRASQDISKYLNWYQQKPDGTVKLLIYHTSR |
| | LHSGVPSRFSGSGSGTDYSLTISNLEQEDIATYFCQQGNTLPYTFGGGTKLEI |
| | TGSTSGSGKPGSGEGSTKGEVKLQESGPGLVAPSQSLSVTCTVSGVSLPDYGV |
| | SWIRQPPRKGLEWLGVIWGSETTYYNSALKSRLTIIKDNSKSQVFLKMNSLQT |
| | DDTAIYYCAKHYYYGGSYAMDYWGQGTSVTVSS |
| Anti-CD20/anti- | SEQ ID NO: 26 |
| CD19 bicistronic | MLLLVTSLLLCELPHPAFLLIPDIQMTQTTSSLSASLGDRVTISCRASQDISK |
| CAR | YLNWYQQKPDGTVKLLIYHTSRLHSGVPSRFSGSGSGTDYSLTISNLEQEDIA |
| | TYFCQQGNTLPYTFGGGTKLEITGGGGSGGGGSGGGGSEVKLQESGPGLVAPS |
| | QSLSVTCTVSGVSLPDYGVSWIRQPPRKGLEWLGVIWGSETTYYNSALKSRLT |
| | IIKDNSKSQVFLKMNSLQTDDTAIYYCAKHYYYGGSYAMDYWGQGTSVTVSSA |
| | AALDNEKSNGTIIHVKGKHLCPSPLFPGPSKPFWVLVVVGGVLACYSLLVTVA |
| | FIIFWVRSKRSRLLHSDYMNMTPRRPGPTRKHYQPYAPPRDFAAYRSRVKFSR |
| | SADAPAYQQGQNQLYNELNLGRREEYDVLDKRRGRDPEMGGKPRRKNPQEGLY |
| | NELQKDKMAEAYSEIGMKGERRRGKGHDGLYQGLSTATKDTYDALHMQALPPR |
| | RAKRSGSGEGRGSLLTCGDVEENPGPMALPVTALLLPLALLLHAARPQLQLQE |
| | SGPGLVKPSETLSLTCTVSGGSISSSSYYWGWIRQPPGKGLEWIGSIYYSGST |
| | YYNPSLKSRVTISVDTSKNQFSLKLSSVTAADTAVYYCARETDYSSGMGYGMD |
| | VWGQGTTVTVSSGSTSGSGKPGSGEGSTKGDIQMTQSPSSLSASVGDRVTITC |
| | RASQSINSYLNWYQQKPGKAPKLLIYAASSLQSGVPSRFSGSGSGTDFTLTIS |
| | SLQPEDFATYYCQQSLADPFTFGGGTKVEIKAAAFVPVFLPAKPTTTPAPRPP |
| | TPAPTIASQPLSLRPEACRPAAGGAVHTRGLDFACDIYIWAPLAGTCGVLLLS |
| | LVITLYCNHRNRFSVVKRGRKKLLYIFKQPFMRPVQTTQEEDGCSCRFPEEEE |
| | GGCELRVKFSRSADAPAYQQGQNQLYNELNLGRREEYDVLDKRRGRDPEMGGK |
| | PRRKNPQEGLYNELQKDKMAEAYSEIGMKGERRRGKGHDGLYQGLSTATKDTY |
| | DALHMQALPPR |
| Anti-CD20/anti- | SEQ ID NO: 27 |
| CD19 bispecific | MLLLVTSLLLCELPHPAFLLIPDIQMTQSPSSLSASVGDRVTITCRASQSINS |
| CAR | YLNWYQQKPGKAPKLLIYAASSLQSGVPSRFSGSGSGTDFTLTISSLQPEDFA |
| | TYYCQQSLADPFTFGGGTKVEIKGGGGSGKPGSGEGGSQLQLQESGPGLVKPS |
| | ETLSLTCTVSGGSISSSSYYWGWIRQPPGKGLEWIGSIYYSGSTYYNPSLKSR |
| | VTISVDTSKNQFSLKLSSVTAADTAVYYCARETDYSSGMGYGMDVWGQGTTVT |
| | VSSGGGGSGKPGSDIQMTQSPSSLSASVGDRVTITCRASQDISKYLNWYQQKP |
| | DQAPKLLIKHTSRLHSGVPSRFSGSGSGTDYTLTISSLQPEDFATYYCQQGNT |
| | LPYTFGQGTKLEIKGGGGSGGGGSGGGGSEVQLVESGGGLVQPGRSLRLSCTA |
| | SGVSLPDYGVSWIRQPPGKGLEWIGVIWGSETTYYNSALKSRFTISRDNSKNT |
| | LYLQMNSLRAEDTAVYYCAKHYYYGGSYAMDYWGQGTLVTVSSAAALDNEKSN |
| | GTIIHVKGKHLCPSPLFPGPSKPFWVLVVVGGVLACYSLLVTVAFIIFWVRSK |
| | RSRLLHSDYMNMTPRRPGPTRKHYQPYAPPRDFAAYRSRVKFSRSADAPAYQQ |
| | GQNQLYNELNLGRREEYDVLDKRRGRDPEMGGKPRRKNPQEGLYNELQKDKMA |
| | EAYSEIGMKGERRRGKGHDGLYQGLSTATKDTYDALHMQALPPR |

A CAR of the present disclosure can include, in addition to the antigen-binding molecule, a hinge, a transmembrane domain, and/or an intracellular domain. In some embodiments, the intracellular domain can include a costimulatory domain and an activation domain.

A hinge may be an extracellular domain of an antigen binding system positioned between the binding motif and the transmembrane domain. A hinge may also be referred to as an extracellular domain or as a “spacer.” A hinge may contribute to receptor expression, activity, and/or stability. A hinge may also provide flexibility to access the targeted antigen. In some embodiments, a hinge domain is positioned between a binding motif and a transmembrane domain.

In some embodiments, the hinge is, is from, or is derived from (e.g., comprises all or a fragment of) an immunoglobulin-like hinge domain. In some embodiments, a hinge domain is from or derived from an immunoglobulin. In some embodiments, a hinge domain is selected from the hinge of IgG1, IgG2, IgG3, IgG4, IgA, IgD, IgE, or IgM, or a fragment thereof.

In some embodiments, the hinge is, is from, or is derived from (e.g., comprises all or a fragment of) CD2, CD3 delta, CD3 epsilon, CD3 gamma, CD4, CD7, CD8.alpha., CD8.beta., CD11a (ITGAL), CD11b (ITGAM), CD11c (ITGAX), CD11d (ITGAD), CD18 (ITGB2), CD19 (B4), CD27 (TNFRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMFI), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, or Toll ligand receptor, or which is a fragment or combination thereof.

In some embodiments, the hinge is, is from, or is derived from (e.g., comprises all or a fragment of) a hinge of CD8 alpha. In some embodiments, the hinge is, is from, or is derived from a hinge of CD28. In some embodiments, the hinge is, is from, or is derived from a fragment of a hinge of CD8 alpha or a fragment of a hinge of CD28, wherein the fragment is anything less than the whole. In some embodiments, a fragment of a CD8 alpha hinge or a fragment of a CD28 hinge comprises an amino acid sequence that excludes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acids at the N-terminus or C-Terminus, or both, of a CD8 alpha hinge, or of a CD28 hinge.

A “transmembrane domain” refers to a domain having an attribute of being present in the membrane when present in a molecule at a cell surface or cell membrane (e.g., spanning a portion or all of a cellular membrane). It is not required that every amino acid in a transmembrane domain be present in the membrane. For example, in some embodiments, a transmembrane domain is characterized in that a designated stretch or portion of a protein is substantially located in the membrane. Amino acid or nucleic acid sequences may be analyzed using a variety of algorithms to predict protein subcellular localization (e.g., transmembrane localization). The programs psort (PSORT.org) and Prosite (prosite.expasy.org) are exemplary of such programs.

A transmembrane domain may be derived either from any membrane-bound or transmembrane protein, such as an alpha, beta or zeta chain of a T-cell receptor, CD28, CD3 epsilon, CD3 delta, CD3 gamma, CD45, CD4, CD5, CD7, CD8, CD8 alpha, CD8beta, CD9, CD11a, CD11b, CD11c, CD11d, CD16, CD22, CD27, CD33, CD37, CD64, CD80, CD86, CD134, CD137, TNFSFR25, CD154, 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD276 (B7-H3), CD29, CD30, CD40, CD49a, CD49D, CD49f, CD69, CD84, CD96 (Tactile), CD5, CEACAM1, CRT AM, cytokine receptor, DAP-10, DNAM1 (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, a ligand that binds with CD83, LIGHT, LIGHT, LTBR, Ly9 (CD229), lymphocyte function-associated antigen-1 (LFA-1; CD1-la/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A; Ly108), SLAMF7, SLP-76, TNF receptor proteins, TNFR2, TNFSF14, a Toll ligand receptor, TRANCE/RANKL, VLA1, or VLA-6, or a fragment, truncation, or a combination thereof.

The intracellular domain (or cytoplasmic domain) comprises one or more signaling domains that, upon binding of target antigen to the binding motif, cause and/or mediate an intracellular signal, e.g., that activates one or more immune cell effector functions (e.g., native immune cell effector functions). In some embodiments, signaling domains of an intracellular domain mediate activation at least one of the normal effector functions of the immune cell. Effector function of a T cell, for example, may be cytolytic activity or helper activity comprising the secretion of cytokines. In some embodiments, signaling domains of an intracellular domain mediate T cell activation, proliferation, survival, and/or other T cell function. An intracellular domain may comprise a signaling domain that is an activating domain. An intracellular domain may comprise a signaling domain that is a costimulatory signaling domain.

Intracellular signaling domains that may transduce a signal upon binding of an antigen to an immune cell are known. For example, cytoplasmic sequences of a T cell receptor (TCR) are known to initiate signal transduction following TCR binding to an antigen (see, e.g., Brownlie et al., Nature Rev. Immunol. 13:257-269 (2013)).

In certain embodiments, suitable signaling domains include, without limitation, those of 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CD11a, CD11b, CD11c, CD11d, CD5, CEACAM1, CRT AM, cytokine receptor, DAP-10, DNAM1 (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, ligand that binds with CD83, LIGHT, LIGHT, LTBR, Ly9 (CD229), Ly108), lymphocyte function-associated antigen-1 (LFA-1; CD1-la/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMFI; CD150; IPO-3), SLAMF4 (CD244; 2B4), SLAMF6 (NTB-A, SLAMF7, SLP-76, TNF receptor proteins, TNFR2, TNFSF14, a Toll ligand receptor, TRANCE/RANKL, VLA1, or VLA-6, or a fragment, truncation, or a combination thereof.

A CAR can also include a costimulatory signaling domain, e.g., to increase signaling potency. See U.S. Pat. Nos. 7,741,465, and 6,319,494, as well as Krause et al. and Finney et al. (supra), Song et al., Blood 119:696-706 (2012); Kalos et al., Sci Transl. Med. 3:95 (2011); Porter et al., N. Engl. J. Med. 365:725-33 (2011), and Gross et al., Annu. Rev. Pharmacol. Toxicol. 56:59-83 (2016). Signals generated through a TCR alone may be insufficient for full activation of a T cell and a secondary or co-stimulatory signal may increase activation. Thus, in some embodiments, a signaling domain further comprises one or more additional signaling domains (e.g., costimulatory signaling domains) that activate one or more immune cell effector functions (e.g., a native immune cell effector function described herein). In some embodiments, a portion of such costimulatory signaling domains may be used, as long as the portion transduces the effector function signal. In some embodiments, a cytoplasmic domain described herein comprises one or more cytoplasmic sequences of a T cell co-receptor (or fragment thereof). Non-limiting examples of such T cell co-receptors comprise CD27, CD28, 4-1BB (CD137), OX40, CD30, CD40, PD-1, ICOS, lymphocyte function-associated antigen-1 (LFA-1), MYD88, CD2, CD7, LIGHT, NKG2C, B7-H3, and a ligand that binds with CD83. An exemplary costimulatory protein has the amino acid sequence of a costimulatory protein found naturally on T cells, the complete native amino acid sequence of which costimulatory protein is described in NCBI Reference Sequence: NP 0.1. In certain instances, a CAR includes a 4-1BB costimulatory domain. In certain instances, a CAR includes a CD28 costimulatory domain. In certain instances, a CAR includes a DAP-10 costimulatory domain.

In some embodiments, the costimulatory signaling domain is a signaling domain of CD28. As shown in the experimental examples, CAR molecules with a CD28 costimulatory signaling domain can particularly benefit from the newly developed, expedited manufacturing process.

In some embodiments, the CAR further includes an ITAM. Examples of ITAM containing primary cytoplasmic signaling sequences that are of particular use in the disclosure include those derived from TCRzeta, FcRgamma, FcRbeta, CD3gamma, CD3delta, CD3epsilon, CD5, CD22, CD79a, CD79b and CD66d. In some embodiments, the ITAM includes CD3 zeta.

In some embodiments, the CAR molecule any anti-CD19 CAR molecule. In one aspect the anti-CD19 CAR includes an extracellular scFv domain, an intracellular and/or transmembrane, portion of a CD28 molecule, an optional extracellular portion of the CD28 molecule, and an intracellular CD3zeta domain as described in WO2015120096 or WO2016191755, each of which is incorporated herein in its entirety.

In certain embodiments, the anti-CD19 CAR may also include additional domains, such as a CD8 extracellular and/or transmembrane region, an extracellular immunoglobulin Fc domain (e.g., lgG1, lgG2, lgG3, lgG4), or one or more additional signaling domains, such as 41 BB, OX40, CD2 CD16, CD27, CD30 CD40, PD-1, ICOS, LFA-1, IL-2 Receptor, Fc gamma receptor, or any other costimulatory domains with immunoreceptor tyrosine-based activation motifs.

In certain embodiments, the cell surface receptor is an anti-CD19 CAR, such as FMC63-28Z CAR or FMC63-CD828BBZ CAR as set forth in Kochenderfer et al., J Immunother. 2009 September; 32(7): 689-702, “Construction and Preclinical Evaluation of an Anti-CD19 Chimeric Antigen Receptor,” the subject matter of which is hereby incorporated by reference for the purpose of providing the methods of constructing the vectors used to produce T cells expressing the FMC63-28Z CAR or FMC63-CD828BBZ CAR.

In some embodiments, the T cell that includes a CAR molecule is Yescarta® (axicabtagene ciloleucel). In some embodiments, the T cell that includes a CAR molecule is Tecartus® (brexucabtagene autoleucel). In some embodiments, the T cell includes one or more CAR molecules that can bind one or more antigenic moieties.

In some embodiments, a pharmaceutical composition is provided that includes a population of engineered lymphocytes produced by the methods described herein. In certain embodiments, the pharmaceutical composition may also include a pharmaceutically acceptable carrier. A pharmaceutically acceptable carrier may be a pharmaceutically acceptable material, composition, or vehicle that is involved in carrying or transporting cells of interest from one tissue, organ, or portion of the body to another tissue, organ, or portion of the body. For example, the carrier may be a liquid or solid filler, diluent, excipient, solvent, or encapsulating material, or some combination thereof. Each component of the carrier must be “pharmaceutically acceptable” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits.

### Treatments and Uses, and Optional Storage

The lymphocytes prepared by the instant methods, or the lymphocyte populations as disclosed herein, can be used for treating various diseases and conditions.

In some embodiments, if the lymphocytes are not immediately used, they can cryopreserved so that they can be used at a later date. Such a method may include a step of washing and concentrating the population of engineered lymphocytes with a diluent solution. In some aspects the diluent solution is normal saline, 0.9% saline, PlasmaLyte A (PL), 5% dextrose/0.45% NaCl saline solution (D5), human serum albumin (HSA), or a combination thereof. In some aspects, HSA may be added to the washed and concentrated cells for improved cell viability and cell recovery after thawing. In another aspect, the washing solution is normal saline and washed and concentrated cells are supplemented with HSA (5%). The method may also include a step of generating a cryopreservation mixture, wherein the cryopreservation mixture includes the diluted population of cells in the diluent solution and a suitable cryopreservative solution. In some aspects, the cryopreservative solution may be any suitable cryopreservative solution including, but not limited to, CryoStor10 (BioLife Solution), mixed with the diluent solution of engineered lymphocytes at a ratio of 1:1 or 2:1.

In certain embodiments, HSA may be added to provide a final concentration of about 1.0-10% HSA in the cryopreserved mixture. In certain embodiments, HSA may be added to provide a final concentration of about 1.0%, about 2.0%, about 3.0%, about 4.0%, about 5.0%, about 6.0%, about 7.0%, about 8.0%, about 9.0%, or about 10.0% HSA in the cryopreserved mixture. In certain embodiments, HSA may be added to provide a final concentration of about 1-3% HSA, about 1-4% HSA, about 1-5% HSA, about 1-7% HSA, about 2-4% HSA, about 2-5% HSA, about 2-6% HSA, or about 2-7% HSA in the cryopreserved mixture. In certain embodiments, HSA may be added to provide a final concentration of about 2.5% HSA in the cryopreserved mixture. For example, in certain embodiments, cryopreservation of a population of engineered T cells may comprise washing cells with 0.9% normal saline, adding HSA at a final concentration of 5% to the washed cells, and diluting the cells 1:1 with CryoStor™ CS10 (for a final concentration of 2.5% HSA in the final cryopreservation mixture). In some embodiments, the method also includes a step of freezing the cryopreservation mixture. In one aspect, the cryopreservation mixture is frozen in a controlled rate freezer using a defined freeze cycle at a cell concentration of between about 1e6 to about 1.5e7 cells per mL of cryopreservation mixture. The method may also include a step of storing the cryopreservation mixture in vapor phase liquid nitrogen.

Methods and uses are also provided, for treating a disease or pathological condition in a subject having the disease or pathological condition. In some embodiments, the method entails administering a therapeutically effective amount or therapeutically effective dose of the engineered lymphocytes to the subject. Pathogenic conditions that may be treated with engineered T cells that are produced by the methods described herein include, but are not limited to, cancer, viral infection, acute or chronic inflammation, autoimmune disease or any other immune-dysfunction.

As referred to herein, a “cancer” may be any cancer that is associated with a surface antigen or cancer marker, including, but not limited to, acute lymphoblastic leukemia (ALL), acute myeloid leukemia (AML), adenoid cystic carcinoma, adrenocortical, carcinoma, AIDS-related cancers, anal cancer, appendix cancer, astrocytomas, atypical teratoid/rhabdoid tumor, central nervous system, B-cell leukemia, lymphoma or other B cell malignancies, basal cell carcinoma, bile duct cancer, bladder cancer, bone cancer, osteosarcoma and malignant fibrous histiocytoma, brain stem glioma, brain tumors, breast cancer, bronchial tumors, burkitt lymphoma, carcinoid tumors, central nervous system cancers, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, embryonal tumors, central nervous system, endometrial cancer, ependymoblastoma, ependymoma, esophageal cancer, esthesioneuroblastoma, ewing sarcoma family of tumors extracranial germ cell tumor, extragonadal germ cell tumor extrahepatic bile duct cancer, eye cancer fibrous histiocytoma of bone, malignant, and osteosarcoma, gallbladder cancer, gastric (stomach) cancer, gastrointestinal carcinoid tumor, gastrointestinal stromal tumors (GIST), soft tissue sarcoma, germ cell tumor, gestational trophoblastic tumor, glioma, hairy cell leukemia, head and neck cancer, heart cancer, hepatocellular (liver) cancer, histiocytosis, hodgkin lymphoma, hypopharyngeal cancer, intraocular melanoma, islet cell tumors (endocrine pancreas), kaposi sarcoma, kidney cancer, langerhans cell histiocytosis, laryngeal cancer, leukemia, lip and oral cavity cancer, liver cancer (primary), lobular carcinoma in situ (LCIS), lung cancer, lymphoma, macroglobulinemia, male breast cancer, malignant fibrous histiocytoma of bone and osteosarcoma, medulloblastoma, medulloepithelioma, melanoma, merkel cell carcinoma, mesothelioma, metastatic squamous neck cancer with occult primary midline tract carcinoma involving NUT gene, mouth cancer, multiple endocrine neoplasia syndromes, multiple myeloma/plasma cell neoplasm, mycosis fungoides, myelodysplastic syndromes, myelodysplastic/myeloproliferative neoplasms, myelogenous leukemia, chronic (CML), Myeloid leukemia, acute (AML), myeloma, multiple, myeloproliferative disorders, nasal cavity and paranasal sinus cancer, nasopharyngeal cancer, neuroblastoma, non-hodgkin lymphoma, non-small cell lung cancer, oral cancer, oral cavity cancer, oropharyngeal cancer, osteosarcoma and malignant fibrous histiocytoma of bone, ovarian cancer, pancreatic cancer, papillomatosis, paraganglioma, paranasal sinus and nasal cavity cancer, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm/multiple myeloma, pleuropulmonary blastoma, pregnancy and breast cancer, primary central nervous system (CNS) lymphoma, prostate cancer, rectal cancer, renal cell (kidney) cancer, renal pelvis and ureter, transitional cell cancer, retinoblastoma, rhabdomyosarcoma, salivary gland cancer, sarcoma, sézary syndrome, small cell lung cancer, small intestine cancer, soft tissue sarcoma, squamous cell carcinoma, squamous neck cancer, stomach (gastric) cancer, supratentorial primitive neuroectodermal tumors, t-cell lymphoma, cutaneous, testicular cancer, throat cancer, thymoma and thymic carcinoma, thyroid cancer, transitional cell cancer of the renal pelvis and ureter, trophoblastic tumor, ureter and renal pelvis cancer, urethral cancer, uterine cancer, uterine sarcoma, vaginal cancer, vulvar cancer, Waldenström macroglobulinemia, Wilms Tumor.

In some aspects, the cancer is a B cell malignancy. Examples of B cell malignancies include, but are not limited to, Non-Hodgkin's Lymphomas (NHL), Diffuse Large B Cell Lymphoma (DLBCL), Small lymphocytic lymphoma (SLL/CLL), Mantle cell lymphoma (MCL), Follicular lymphoma (FL), Marginal zone lymphoma (MZL), Extranodal (MALT lymphoma), Nodal (Monocytoid B-cell lymphoma), Splenic, Diffuse large cell lymphoma, B cell chronic lymphocytic leukemia/lymphoma, Burkitt's lymphoma and Lymphoblastic lymphoma.

As referred to herein, a “viral infection” may be an infection caused by any virus which causes a disease or pathological condition in the host. Examples of viral infections that may be treated with the engineered T cells that are produced by the methods described herein include, but are not limited to, a viral infection caused by an Epstein-Barr virus (EBV); a viral infection caused by a hepatitis A virus, a hepatitis B virus or a hepatitis C virus; a viral infection caused by a herpes simplex type 1 virus, a herpes simplex type 2 virus, or a herpes simplex type 8 virus, a viral infection caused by a cytomegalovirus (CMV), a viral infection caused by a human immunodeficiency virus (HIV), a viral infection caused by an influenza virus, a viral infection caused by a measles or mumps virus, a viral infection caused by a human papillomavirus (HPV), a viral infection caused by a parainfluenza virus, a viral infection caused by a rubella virus, a viral infection caused by a respiratory syncytial virus (RSV), or a viral infection caused by a varicella-zostser virus. In some aspects, a viral infection may lead to or result in the development of cancer in a subject with the viral infection (e.g., HPV infection may cause or be associated with the development of several cancers, including cervical, vulvar, vaginal, penile, anal, oropharyngeal cancers, and HIV infection may cause the development of Kaposi's sarcoma).

Examples of chronic inflammation diseases, autoimmune diseases or any other immune-dysfunctions that may be treated with the engineered T cells produced by the methods described herein include, but are not limited to, multiple sclerosis, lupus, and psoriasis.

The term “treat,” “treating” or “treatment” as used herein with regard to a condition or disease may refer to preventing a condition or disease, slowing the onset or rate of development of the condition or disease, reducing the risk of developing the condition or disease, preventing or delaying the development of symptoms associated with the condition or disease, reducing or ending symptoms associated with the condition or disease, generating a complete or partial regression of the condition or disease, or some combination thereof.

A “therapeutically effective amount” or a “therapeutically effective dose” is an amount of engineered lymphocytes that produce a desired therapeutic effect in a subject, such as preventing or treating a target condition or alleviating symptoms associated with the condition by killing target cells. The most effective results in terms of efficacy of treatment in a given subject will vary depending upon a variety of factors, including but not limited to the characteristics of the engineered lymphocytes (including longevity, activity, pharmacokinetics, pharmacodynamics, and bioavailability), the physiological condition of the subject (including age, sex, disease type and stage, general physical condition, responsiveness to a given dosage, and type of medication), the nature of any pharmaceutically acceptable carrier or carriers in any composition used, and the route of administration. A therapeutically effective dose of engineered lymphocytes also depends on the cell surface receptor that is expressed by the lymphocytes (e.g., the affinity and density of the cell surface receptors expressed on the cell), the type of target cell, the nature of the disease or pathological condition being treated, or a combination of both.

As shown in the examples, the engineered lymphocytes prepared by the instant process have greatly increased in vivo efficacy and thus a much lower dose is required, as compared to the conventional technology.

Therefore, in some aspects, a therapeutically effective dose of engineered lymphocytes is fewer than about 2 million engineered lymphocytes per kilogram of body weight of the subject in need of treatment (cells/kg). Therefore, in some aspects, a therapeutically effective dose of engineered lymphocytes is from about 10,000 to about 1,500,000 engineered lymphocytes/kg. In certain embodiments, the therapeutically effective dose is about 20,000 to about 1,200,000 million engineered lymphocytes/kg. In certain embodiments, the therapeutically effective dose is about 20,000 to about 1,000,000 million engineered lymphocytes/kg. In certain embodiments, the therapeutically effective dose is about 20,000 to about 500,000 million engineered lymphocytes/kg. In certain embodiments, the therapeutically effective dose is about 20,000 to about 400,000 million engineered lymphocytes/kg. In certain embodiments, the therapeutically effective dose is about 40,000 to about 400,000 million engineered lymphocytes/kg. In certain embodiments, the therapeutically effective dose is about 50,000 to about 200,000 million engineered lymphocytes/kg. In certain embodiments, the therapeutically effective dose is about 50,000 to about 100,000 million engineered lymphocytes/kg.

In some embodiments, the T cells administered are Yescarta® (axicabtagene ciloleucel). In some embodiments, the T cells administered are Tecartus® (brexucabtagene autoleucel).

An embodiment of the disclosure is related to a method for predicting a likelihood of a complete response in a patient to an immunotherapy, comprising: determining a period of time from a leukapheresis step of said patient to an administration of said immunotherapy to said patient; grouping said patient into one of a plurality of groups based on said determining a period of time, said plurality of groups comprising: a first group characterized by a period of up to 28 days (e.g., 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, or 6 days) from said leukapheresis step to said administration of said immunotherapy to said patient; a second group characterized by a period of time of between 28 days to 40 days (e.g., 28 days, 29 days, 30 days, 31 days, 32 days, 33 days, 34 days, 35 days, 36 days, 37 days, 38 days, or 39 days) from said leukapheresis step to said administration of said immunotherapy to said patient; and a third group characterized by a period of time of at least 40 days (e.g., 40 days, 41 days, 42 days, 43 days, 44 days, 45 days, 46 days, 47 days, 48 days, 49 days, or 50 days) from said leukapheresis step to said administration of said immunotherapy to said patient; and determining said likelihood of a complete response in said patient based at least in part on which of said plurality of groups said patient is grouped into, wherein said patient has at least about a 55% likelihood of a complete response if said patient is grouped within said first group or said second group, and wherein said patient has at least about a 42% likelihood of a complete response if said patient is grouped within said third group.

As used herein, the term up to 28 days can mean less than (<) 28 days (e.g., 27 days, 26 days, 20 days, 10 days, or 5 days).

As used herein, the term between 28 days to 40 days can mean greater than or equal (≥) to 28 days to less than (<) 40 days.

As used herein, the term at least 40 days can mean greater than or equal to (≥) 40 days.

In some embodiments of the disclosure, said patient has about a 60% likelihood of a complete response if said patient is grouped within said first group or said second group.

An embodiment of the disclosure relates to a method for predicting an overall survival rate in a patient to an immunotherapy, comprising: determining a period of time from a leukapheresis step of said patient to an administration of said immunotherapy to said patient; grouping said patient into one of a plurality of groups based on said determining a period of time, said plurality of groups comprising: a first group characterized by a period of up to 28 days from said leukapheresis step to said administration of said immunotherapy to said patient; a second group characterized by a period of time of between 28 days to less than 40 days from said leukapheresis step to said administration of said immunotherapy to said patient; and a third group characterized by a period of time of at least 40 days from said leukapheresis step to said administration of said immunotherapy to said patient; and determining said overall survival rate in said patient based at least in part on which of said plurality of groups said patient is grouped into, wherein said patient has at least about a 49% overall survival rate if said patient is grouped within said first group, wherein said patient has at least about a 48% overall survival rate if said patient is grouped within said second group, and wherein said patient has at least about a 30% overall survival rate if said patient is grouped within said third group.

An embodiment of the disclosure relates to a method for predicting a risk of thrombocytopenia in a patient receiving an immunotherapy, comprising: determining a period of time from a leukapheresis step of said patient to an administration of said immunotherapy to said patient; grouping said patient into one of a plurality of groups based on said determining a period of time, said plurality of groups comprising: a first group characterized by a period of time of up to 28 days from said leukapheresis step to said administration of said immunotherapy to said patient; a second group characterized by a period of time of between 28 days to 40 days from said leukapheresis step to said administration of said immunotherapy to said patient; and a third group characterized by a period of time of at least 40 days from said leukapheresis step to said administration of said immunotherapy to said patient; and determining said risk of thrombocytopenia in said patient based at least in part on which of said plurality of groups said patient is grouped into, wherein said patient has about an 18% risk of thrombocytopenia if said patient is grouped within said first group, wherein said patient has about a 25% risk of thrombocytopenia if said patient is grouped within said second group, and wherein said patient has about a 34% risk of thrombocytopenia if said patient is grouped within said third group.

An embodiment of the disclosure relates to a method for predicting a life expectancy and quality-adjusted life years in a patient that has received an immunotherapy, comprising: determining a period of time from a leukapheresis step of said patient to an administration of said immunotherapy to said patient, wherein the period of time is a short period or a long period; assigning a probability of successful infusion based on the period of time; entering the patient information into a survival model to determine the life expectancy and the quality-adjusted life years of the patient.

In some embodiments, the model described herein may be a decision tree model with outcomes associated with long or short V2VT.

In some embodiments, the model inputs may comprise: a long V2VT, a short V2VT, a probability of infusion, lifetime outcomes (non-infused patients), lifetime outcomes (infused patients), efficacy of long vs short V2VT for infused patients, and/or QALYs.

In some embodiments, the model outputs may comprise life years (LYs) and/or Quality Adjusted Life Years (QALYs).

In some embodiments, the model described herein may comprise decision gates/node and probability nodes. A decision gate may represent a point at which a decision is made (e.g., long V2VT or short V2VT). A probability node may represent the probabilities of certain results (e.g., infused or not infused). Graph survival projections may be used to represent modeled lifetime outcomes based on data inputs.

As used herein, the term “life expectancy” refers to the number of years a subject or patient can expect to live. In some embodiments, a gain in life expectancy may be measured from when the patient is informed that they will receive an immunotherapy. In some embodiments, a gain in life expectancy may be measured from when the patient is infused with an immunotherapy.

As used herein, the term “quality-adjusted life years” or “QALY” refers to a measure of disease burden or health outcomes, including both the quality and quantity of life. In some embodiments, a gain in QALY may be measured from when the patient is informed that they will receive an immunotherapy. In some embodiments, a gain in QALY may be measured from when the patient is infused with an immunotherapy.

As used herein, the term “short period” may refer to a period of time from a leukapheresis step of a patient to an administration of an immunotherapy to the patient (e.g., a vein-to-vein time) of 24 days or less.

As used herein, the term “long period” may refer to a period of time from a leukapheresis step of a patient to an administration of an immunotherapy to the patient (e.g., a vein-to-vein time) of 37 days or more, 54 days or more, or between 37 days and 54 days.

In some embodiments, a short period of time may indicate a gain in life expectancy and/or quality-adjusted life years of the patient to be greater than about 5 years (e.g., about 5.5 years, 6 years, 6.5 years, 7 years, 7.5 years, 8 years, 8.5 years, 9 years, 9.5 years, or 10 years or more).

In some embodiments, a long period of time may indicate a gain in life expectancy and/or a gain in quality-adjusted life years of the patient to be less than about 5 years (e.g., about 4.5 years, 4 years, 3.5 years, 3 years, 2.5 years, 2 years, 1.5 years, or 1 year or less).

In an embodiment of the disclosure, the immunotherapy comprises one or more CARs recognizing one or more tumor antigens.

In an embodiment of the disclosure, the immunotherapy is axicabtagene ciloleucel or brexucabtagene autoleucel.

In an embodiment of the disclosure, the patient has relapsed or refractory (r/r) large B-cell lymphoma (LBCL).

The following examples are intended to illustrate various embodiments of the invention.

As such, the specific embodiments discussed are not to be construed as limitations on the scope of the invention. For example, although the Examples below are directed to T cells transduced with an anti-CD19 chimeric antigen receptor (CAR), one skilled in the art would understand that the methods described herein may apply to T cells transduced with any CAR or TCR. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### Example 1

Compared with other CAR T-cell products, axicabtagene ciloleucel (axi-cel) had a shorter median wait time from leukapheresis to infusion, referred to as vein-to-vein time (real-world: axi-cel, 28 d vs tisagenlecleucel, 45 d; clinical trial: lisocabtagene maraleucel, 36-37 d; Riedell et al. Transplant Cell Ther 2022; Abramson et al. Lancet 2020). A study based on the JULIET trial suggested that reduced wait time is associated with increased efficacy (Chen et al. Value Health 2022). This example assessed the real-world impact of vein-to-vein time on outcomes of axi-cel in r/r LBCL.

### Methods

A total of 1383 patients from 78 US centers treated with commercial axi-cel for r/r LBCL were identified from a non-interventional post-authorization safety study using the Center for International Blood and Marrow Transplant Research (CIBMTR) registry. Patients with the following were excluded: primary central nervous system lymphoma or lymphoma other than LBCL, prior non-transplant cellular therapy, missing data on comorbidity (Sorror et al. Blood 2005), unknown or outlying date of leukapheresis (≤2 d before lymphodepleting [LD] chemotherapy or ≥144 d before infusion), or no follow-up.

Effectiveness outcomes were overall and complete response rates (ORR and CR), duration of response (DOR), and progression-free and overall survival (PFS and OS). Adverse events of interest included cytokine release syndrome (CRS) (Lee 2014 criteria), immune effector cell-associated neurotoxicity syndrome (ICANS) (ASTCT criteria), prolonged neutropenia and thrombocytopenia. Odds ratios (ORs) and hazard ratios (HRs) were estimated using logistic and Cox regressions after adjustment of key prognostic factors such as age, comorbidities, ECOG performance status, disease characteristics at diagnosis, and bridging therapy. Adjusted curves were generated based on the direct adjusted survival function (Makuch J Chronic Dis 1982).

### Results

Overall, median vein-to-vein time (from leukapheresis to infusion) for axi-cel was 27 d (interquartile range [IQR], 26-32 d), including a median of 5 d (IQR, 5-5 d) from start of LD chemotherapy to infusion. Vein-to-vein times were consistent regardless of the following baseline characteristics: disease histology, sex, race, ethnicity, ECOG performance status before infusion, or chemo-sensitivity (Table 2).

Pts with shorter vein-to-vein times appeared to be younger and less likely to have comorbidities (Table 3). Patients with vein-to-vein time of 40 d were more heavily pretreated and more likely to receive bridging therapy.

At a median follow-up of 24.2 months, better outcomes were observed in patients with shorter vein-to-vein times. CR rates were 60%, 61%, and 50% (ORR 77%, 77%, and 70%) for patients with vein-to-vein time of <28 d, ≥28 to <40 d, and ≥40 d, respectively. OS at 24 months was 53% for patients with vein-to-vein time of both <28 d and ≥28 to <40 d, vs 38% for those with ≥40 d wait time. After other key prognostic factors were adjusted, patients with vein-to-vein time of ≥40 d had significantly lower CR rate and OS compared with patients with <28 d (OR 0.61 [95% CI 0.42-0.90] for CR; HR 1.33 [95% CI 1.05-1.70] for OS) and ≥28 to <40 d wait time (OR 0.66 [95% CI 0.45-0.97] for CR; HR 1.36 [95% CI 1.06-1.74] for OS).

Adjusted progression-free survival (PFS), overall survival (OS), and duration of response (DOR) analyses based a stratified Cox model (Sorror, M L, et al. Blood. 2005; 106(8): 2912-2919; Chang I M, et al. J Chronic Dis. 1982; 35:669-674) were conducted to balance differences in baseline characteristics. Sensitivity analyses comparing outcomes for patients with vein-to-vein time <36 days versus ≥36 days were also carried out to assess the validity of the vein-to-vein time categorization used in the primary analysis.

Among patients who achieved CR/partial response (PR) as best response, DOR at 12 months was 61% for patients with vein-to-vein time of <28 days, 60% for vein-to-vein time of ≥28 to <40 days, and 61% for vein-to-vein time of ≥40 days. Sensitivity analyses for DOR were consistent with the primary analyses.

Adjusted PFS and OS at 24 months appeared lower for patients with vein-to-vein time ≥40 days versus patients with vein-to-vein time of <28 days or ≥28 days to <40 days. Sensitivity analyses for OS and PFS were consistent with the primary analyses, with OS being significantly shorter for patients with vein-to-vein time ≥36 days compared with patients with vein-to-vein time <36 days (hazard ratio [HR], 1.25 [95% CI, 1.02-1.53]).

CRS of any grade or Grade 3 and prolonged neutropenia were consistent regardless of vein-to-vein time. Patients with vein-to-vein time of <28 d had more ICANS of any grade compared with those with ≥28 to <40 d wait time (OR 1.34 [95% CI 1.06-1.71]), while ICANS of Grade 3 was not significantly different between the two groups (Table 2). Among patients alive at Day 30, those with vein-to-vein time of ≥28 to <40 d and ≥40 d had higher rates of prolonged thrombocytopenia compared with those with ≤28 d wait time (OR 1.44 [95% CI 1.07-1.92] and 1.95 [95% CI 1.29-2.95], respectively).

The CRS grade was based on criteria from Lee, D. W., et al. Blood. 2014; 124(2):188-195. The ICANS grade was based on criteria from Lee, D. W., et al. Biol Blood Marrow Transplant. 2019; 25(4):625-638.

Most CRS and ICANS were resolved by 21 days from onset regardless of vein-to-vein time. Cumulative incidence of CRS resolution by 21 days from onset were 92%, 92%, and 94% for patients with vein-to-vein time of <28 d, ≥28 to <40 d, and ≥40 d, respectively. Cumulative incidence of ICANS resolution by 21 days from onset were 79%, 76%, and 64% for patients with vein-to-vein time of <28 d, ≥28 to <40 d, and ≥40 d, respectively.

Sensitivity analyses for the safety outcomes were consistent with the primary analyses.

Multivariable results for the sensitivity analysis comparing patients with a vein-to-vein time ≥36 days to patients with a vein-to-vein time <36 days are shown in Table 4.

In this real-world analysis, most patients with r/r LBCL received axi-cel infusion within 5 weeks after apheresis. Shorter vein-to-vein time was associated with a favorable CR rate, OS, and reduced risk of prolonged thrombocytopenia even after adjustment of key prognostic factors; however, ICANS of any grade may be higher. Overall, these findings highlight the importance of shortening vein-to-vein time in patients treated with axi-cel.

TABLE 2
Baseline Characteristics by Vein-to-Vein Time

| | Vein-to-Vein Time | | | | | |
|---|---|---|---|---|---|---|
| | | | ≥28 | | | |
| | <28 d | | to <40 d | | ≥40 d | |
| | (N = 697) | | (N = 533) | | (N = 153) | |
| Age ≥65 years at | 239 | (34) | 217 | (41) | 65 | (42) |
| infusion, n (%) | | | | | | |
| Male sex, n (%) | 455 | (65) | 348 | (65) | 91 | (59) |
| Black or African | 28 | (4) | 34 | (6) | 9 | (6) |
| American, n (%) | | | | | | |
| Hispanic or Latino, n (%) | 76 | (11) | 56 | (11) | 18 | (12) |
| High grade B-cell | 115 | (16) | 96 | (18) | 20 | (13) |
| lymphoma, n (%) | | | | | | |
| Double/triple hit, n (%)^{a} | 106 | (26) | 87 | (29) | 18 | (20) |
| ECOG PS ≥2 at | 35 | (5) | 20 | (4) | 9 | (6) |
| infusion, n (%) | | | | | | |
| Chemoresistant prior to | 469 | (67) | 355 | (67) | 101 | (66) |
| infusion, n (%) | | | | | | |
| No. of prior lines ≥3, n (%)^{a,b} | 485 | (71) | 361 | (70) | 118 | (82) |
| Use of bridging | 132 | (20) | 109 | (22) | 65 | (46) |
| therapy, n (%)^{a} | | | | | | |
| Any comorbidities, n (%)^{c} | 479 | (69) | 382 | (72) | 125 | (82) |
| Year of infusion: ≤2018, | 210 | (30) | 155 | (29) | 30 | (20) |
| n (%) | | | | | | |
| Year of infusion: 2019, n (%) | 324 | (46) | 252 | (47) | 69 | (45) |
| Year of infusion: 2020, n (%) | 163 | (23) | 126 | (24) | 54 | (35) |

^{a}Percentages were based on non-missing cases.
^{b}Not including prior transplant.
^{c}Defined based on the hematopoietic cell transplant-specific comorbidity index (Sorror, M L, et al. Blood. 2005; 106(8): 2912-2919). ECOG PS, Eastern Cooperative Oncology Group performance status.

TABLE 3
Baseline characteristics, effectiveness, and safety outcomes of axi-
cel by vein-to-vein time (time from leukapheresis to infusion)

| | Vein-to-Vein Time Category | | | | | |
|---|---|---|---|---|---|---|
| | Descriptive, % (95% CI) | | | Multivariable Results, OR/HR (95% CI)^{1,2} | | |
| | | ≥28 | | ≥28 | | ≥40 d |
| Characteristic or | <28 d | to <40 d | ≥40 d | to <40 d | ≥40 d | vs ≥ 28 |
| Outcome Measure | (N = 697) | (N = 533) | (N = 153) | vs <28 d | vs <28 d | to < 40 d |

Baseline characteristics

| | | | |
|---|---|---|---|
| Age <65 years at | 66% | 59% | 58% |
| infusion | | | |
| Presence of any | 69% | 72% | 82% |
| comorbidities³ | | | |
| ≥3 prior lines of | 71% | 70% | 82% |
| therapy⁴ | | | |
| Use of bridging | 20% | 22% | 46% |
| therapy⁵ | | | |

Effectiveness outcomes

| | | | | | | |
|---|---|---|---|---|---|---|
| ORR | 77% | 77% | 70% | 0.96 | 0.66 | 0.69 |
| | (74%- | (73%- | (62%- | (0.72- | (0.43- | (0.45- |
| | 80%) | 80%) | 77%) | 1.27) | 1.00) | 1.06) |
| CR | 60% | 61% | 50% | 0.93 | 0.61 | 0.66 |
| | (56%- | (56%- | (42%- | (0.73- | (0.42- | (0.45- |
| | 64%) | 65%) | 59%) | 1.20) | 0.90)* | 0.97)* |
| DOR at 12 | 62% | 60% | 59% | 1.02 | 1.25 | 1.22 |
| months⁶ | (57%- | (55%- | (49%- | (0.83- | (0.92- | (0.90- |
| | 66%) | 65%) | 68%) | 1.26) | 1.69) | 1.66) |
| PFS at 24 months⁷ | 43% | 39% | 30% | 1.02 | 1.25 | 1.23 |
| | (39%- | (35%- | (23%- | (0.88- | (1.00- | (0.98- |
| | 47%) | 44%) | 38%) | 1.19) | 1.57)* | 1.54) |
| OS at 24 months | 53% | 53% | 38% | 0.98 | 1.33 | 1.36 |
| | (49%- | (48%- | (30%- | (0.83- | (1.05- | (1.06- |
| | 57%) | 57%) | 47%) | 1.17) | 1.70)* | 1.74)* |

Safety outcomes

| | | | | | | |
|---|---|---|---|---|---|---|
| CRS of grade ≥3⁸ | 8% | 8% | 10% | 0.94 | 1.43 | 1.53 |
| | (6%- | (6%- | (6%- | (0.61- | (0.78- | (0.82- |
| | 10%) | 11%) | 16%) | 1.44) | 2.63) | 2.85) |
| ICANS of grade ≥3⁸ | 27% | 24% | 27% | 0.84 | 0.95 | 1.13 |
| | (24%- | (21%- | (20%- | (0.64- | (0.63- | (0.74- |
| | 31%) | 28%) | 35%) | 1.10) | 1.44) | 1.73) |
| Prolonged | 6% | 7% | 9% | 1.35 | 1.40 | 1.04 |
| neutropenia⁹ | (4%- | (5%- | (5%- | (0.84- | (0.71- | (0.52- |
| | 8%) | 10%) | 15%) | 2.17) | 2.77) | 2.08) |
| Prolonged | 18% | 25% | 34% | 1.44 | 1.95 | 1.36 |
| | (16%- | (21%- | (27%- | (1.07- | (1.29- | (0.89- |
| thrombocytopenia⁹ | 22%) | 28%) | 43%) | 1.92)* | 2.95)* | 2.06) |

¹Covariates for step-wise selection and multivariable adjustment: age, sex, race, ethnicity, ECOG performance score prior to infusion, comorbidities (including pulmonary, cardiac/cerebrovascular/heart valve disease, hepatic, and renal), histologic transformation, disease characteristics at initial diagnosis (including double/triple hit, disease stage, elevated lactate dehydrogenase and >1 extranodal involvements), chemo-sensitivity prior to infusion, number of prior lines of therapy, prior HCT, year of infusion, time from initial diagnosis to infusion and use of bridging therapy.
²OR for ORR, CR, and safety outcomes; HR for DOR, PFS, and OS.
³Sorror et al. Blood. 2005.
⁴Among evaluable cases; n = 1340.
⁵Among evaluable cases; n = 1311.
⁶DOR was evaluated among pts achieve initial CR/PR and censored at subsequent cellular therapy or HCT.
⁷PFS was censored at subsequent cellular therapy or HCT.
⁸CRS and ICANS were based on 100-d follow-up reporting.
⁹Prolonged neutropenia and thrombocytopenia were evaluated among pts who were alive at Day 30.
*Statistically significant at a confidence level of 0.05.
CI, confidence interval;
CR, complete response;
CRS, cytokine release syndrome;
DOR, duration of response;
ECOG, Eastern Cooperative Oncology Group;
ICANS, immune effector cell-associated neurotoxicity syndrome;
HCT, hematopoietic stem cell transplant;
HR, hazard ratio;
OS, overall survival;
OR, odds ratio;
ORR, overall response rate;
PFS, progression-free survival;
PR, partial response

TABLE 4
Effectiveness and safety outcomes in patients with a
vein-to-vein time ≥36 days versus <36 days

| | Multivariable Results, |
|---|---|
| | OR/HR (95% CI)^{1,2} |
| ORR | OR 0.74 (0.52-1.05) |
| CR | OR 0.72 (0.52-0.98) |
| DOR | HR 1.23 (0.95-1.58) |
| PFS | HR 1.17 (0.97-1.42) |
| OS | HR 1.25 (1.02-1.53) |
| CRS of grade ≥3 | OR 1.45 (0.87-2.41) |
| ICANS of grade ≥3 | OR 0.92 (0.65-1.30) |
| Prolonged neutropenia | OR 1.04 (0.58-1.87) |
| Prolonged thrombocytopenia | OR 1.62 (1.15-2.28) |

### Example 2

Chimeric antigen receptor (CAR) T-cell therapies have revolutionized the treatment of hematological cancers. However, production requires a complex multistep process from leukapheresis, manufacturing, transport, and storage before final infusion. This time is known as the vein-to-vein time (V2VT) during which the patient's condition may deteriorate, thus highlighting the potential importance of V2VT on patients outcome. This modelling study was designed to compare potential outcomes of a ‘long’ vs ‘short’ V2VT for relapsed/refractory large B-cell lymphoma (r/r LBCL) patients treated with CAR T-cell therapy in the 3L+ setting.

The objective of this study was to compare the lifetime outcomes of a hypothetical cohort of patients receiving CAR T therapy for the treatment of r/r LBCL in the 3L+ setting, but with differing V2VTs.

### Methods

A hypothetical cohort of patients enter a decision tree model at leukapheresis, they are then assigned a probability of successful infusion based on V2VT.

Patients then enter a partition survival model which, estimates life-years (LYs) and quality-adjusted life years (QALYs) over a lifetime horizon, which were based on real world axi-cel OS data. Other CAR T efficacy data were not included to provide conservative estimates and to avoid confounding. The model was informed by published literature, including studies that investigated how many patients ultimately underwent infusion versus the time elapsed from leukapheresis (based on the ZUMA-1, JULIET, and TRANSCEND-NHL-001 studies), the relationship between V2VT and survival (Locke et al. [2022]), as well as studies that investigated differences in survival for infused versus non-infused patients (Kuhl et al., [2022] and Bachy et al., [2022]) (Table 5).

An epidemiological model was used to extrapolate results to CAR T-eligible US patients. Based on the V2VT assignment, the probability of successful infusion was determined and applied to a decision tree. Survival post-V2VT was estimated separately for those successfully infused versus those not. Finally, scenario analyses were performed to assess the robustness of results to key assumptions.

Three hypothetical V2VT cases were explored, based on the reported V2VT of the best available evidence: 54 days (tisa-cel median V2VT; JULIET), 37 days (liso-cel median V2VT; TRANSCEND-NHL-001), and 24 days (axi-cel median V2VT; ZUMA-1).

TABLE 5
Data Inputs Used in the Model

| Input | Source |
|---|---|
| Definition of ‘long’ or | Median V2VT reported from pivotal CAR T trials in 3L + |
| ‘short’ | LBCL setting: ZUMA-1 (24 days) (Neelapu et al. N Engl J |
| | Med. 2017; 377(26): 2531-2544), JULIET (54 days) (Schuster |
| | S J, et al. N Engl J Med. 2019; 380(1): 45-56), and |
| | TRANSCEND-NHL (37 days) (Abramson et al. Lancet. |
| | 2020; 396(10254): 839-852) |
| Probability of infusion | Linear regression model based on proportion infused and |
| | median V2VT (Neelapu et al. N Engl J Med. |
| | 2017; 377(26): 2531-2544; Schuster S J, et al. N Engl J Med. |
| | 2019; 380(1): 45-56; Abramson et al. Lancet. |
| | 2020; 396(10254): 839-852). |
| Lifetime outcomes - non- | Mixture cure modelling using survival data from real-world |
| infused patients | evidence for base-case analysis (Locke et al., Blood 2022; 140 |
| | (Supplement 1): 7512-7515) and sensitivity analysis (Kuhnl |
| | et al., Br J Haematol. 2022; 198(3): 492-502). |
| Lifetime outcomes - | Mixture cure modelling using survival data from real-world |
| infused patients | evidence for base-case analysis (Locke et al., Blood 2022; 140 |
| | (Supplement 1): 7512-7515) and sensitivity analysis (Kuhnl |
| | et al., Br J Haematol. 2022; 198(3): 492-502; Bachy et al., Nat |
| | Med. 2022; 28(10): 2145-2154). |
| Efficacy of ‘long’ vs ‘short’ | Hazard ratio (1.25) applied to the infused patient outcomes |
| V2VT for infused patients | ((Locke et al., Blood 2022; 140 (Supplement 1): 7512-7515) |
| Quality-adjusted life years | An average of utility weights (0.6845) for progression-free |
| (QALYs) | and progressed patients (National Institute for Health and Care |
| | Excellence. Public committee papers: Axicabtagene ciloleucel |
| | for treating diffuse large B-cell lymphoma and primary |
| | mediastinal B-cell lymphoma after 2 or more systemic |
| | therapies [TA559], 2019, www.nice.org.uk/guidance/ta55) |

In order to isolate the effect of V2VT on survival, efficacy outcomes were assumed to be equivalent across the different CAR T therapies and efficacy data for axi-cel were applied in the model due to the limited availability of relevant published data to inform model inputs. A range of sensitivity analyses were conducted in order to test the robustness of the results.

Finally, the per-patient results were scaled using an epidemiology model to estimate the population outcomes if all CAR T eligible patients in the US had a reduced V2VT. Epidemiology estimates were taken from NICE resource impact report (National Institute for Health and Care Excellence. Resource impact report: Axicabtagene ciloleucel for treating diffuse large Bcell lymphoma and primary mediastinal large B-cell lymphoma after 2 or more systemic therapies, March 2023) but modified for the US population. An estimated 2,700 patients were assumed to be eligible for CAR T in the US.

### Results

Survival projections were modelled for the three hypothetical patient cohorts with different V2VTs (case 1: 24 days, case 2: 54 days, and case 3: 37 days).

Median overall survival for the three hypothetical patient cohorts were 19.5 months, 8.5 months and 10.5 months for case 1, 2, and 3 respectively.

Reducing V2VT from 54 days (tisa-cel median V2VT; JULIET) to 24 days (axi-cel median V2VT; ZUMA-1) led to a 3-year gain in life expectancy (4.2 vs 7.7 LYs), and an additional 2 QALYs (2.9 vs 5.3) per patient. This translates to 9,328 additional LYs and 6,385 additional QALYs every year, if all ≈2,710 eligible patients in the US received a ‘short’ V2VT (24-day), rather than a long (54 day) V2VT. See Table 6. Using a smaller difference in V2VT (24 vs 37 days [liso-cel median V2VT; TRANSCEND-NHL-001]) produced 2.6 and 1.8 additional LYs and QALYs, respectively, equating to population level gains of 7,040 LYs and 4,819 QALYs. Outcomes were consistently positive across all sensitivity analyses.

TABLE 6
Base case per patient and population level results

| | Total per- | | Incremental per | | Incremental US | |
|---|---|---|---|---|---|---|
| | patient | | patient | | population | |
| | outcomes | | outcomes | | outcomes | |
| | LYs | QALYs | LYs | QALYs | LYs | QALYs |
| ‘Short’ V2VT: 24 days | 7.68 | 5.26 | — | — | — | — |
| ‘Long’ V2VT: 54 days | 4.24 | 2.90 | 3.44 | 2.36 | 9,328 | 6,385 |

Extensive sensitivity analyses were performed and all analyses showed that a shorter V2VT time led to an improvement in outcomes (Table 7).

The sensitivity analyses demonstrates that outcomes are largely driven by the post-infusion outcomes as a function of V2VT, and the probability of infusion as a function of V2VT parameters.

TABLE 7
Sensitivity analyses incremental results on LYs

| | | LY: | LY: |
|---|---|---|---|
| | | Per- | US |
| Scenario number and description | | patient | population |
| Base case results | | 3.44 | 9,328 |
| 1 | Probability of infusion not affected by V2VT | 1.98 | 5,375 |
| 2 | Post-infusion survival not affected by V2VT | 0.94 | 2,537 |
| | (Bachy et al. 2022) | | |
| 3 | Switch non-infused survival source (Kuhnl et | 3.43 | 9,305 |
| | al. 2022) | | |
| 4 | Switch HR cut-offs (to <28 vs ≥28 to <40 | 3.71 | 10,050 |
| | vs ≥40) | | |
| 5 | Change ‘long’ V2VT to ‘short’ V2VT | 2.60 | 7,040 |
| | (Neelapu et al. 2017; Schuster et al. 2019) | | |
| 6 | Change ‘short’ V2VT to 30 days | 3.02 | 8,174 |

Sensitivity analyses yielded per-patient QALY gains in the range of 0.94 (post-infusion survival not affected by V2VT) to 3.71 (increasing the granularity the cut-offs for V2VT categorization [<28 vs ≥28 to <40 vs ≥40, instead of <36 days vs ≥36 days]).

This study is the first to quantify potential lifetime survival and QALY outcomes for r/r LBCL patients treated with CAR T cells in the 3L+ setting associated with reducing V2VT utilizing currently available evidence.

In the real-world setting, there are multiple factors that can impact V2VT for patients receiving CAR T, and delays during this multi-step process may impact patient outcomes. This study synthesizes publicly available real-world data to demonstrate a potential difference in survival outcomes based on V2VT. The modelling described herein demonstrated that outcomes were primarily determined by a higher probability of reaching infusion and, subsequently, improved outcomes were demonstrated for infused patients compared with those not infused. Outcomes were improved across a range of tested sensitivity analyses.

As is common in modelling studies, a number of key assumptions were made, including generalisation of the HR (hazard ratio); the absence of a formal interrogation of bridging therapy on outcomes; and assuming no difference in efficacy across therapies. However, a range of sensitivity analyses were preformed to test the impact of these assumptions.

V2VT may be an important predictor of outcomes in R/R LBCL and aiming for short manufacturing, product release, shipment and infusion are key to further improve CAR T cell outcomes. This study demonstrates that moderate difference in V2VT may lead to pronounced effects on life expectancy.

While a number of embodiments have been described, it is apparent that the disclosure and examples may provide other embodiments that utilize or are encompassed by the compositions and methods described herein. Therefore, it will be appreciated that the scope of is to be defined by that which may be understood from the disclosure and the appended claims rather than by the embodiments that have been represented by way of example.

1. A method for preparing lymphocytes having improved efficacy and/or reduced adverse effects in treating cancer, the method comprising acquiring lymphocytes from a patient through apheresis;
incubating the lymphocytes with a polynucleotide vector to transduce the lymphocytes to produce transduced lymphocytes;
culturing the transduced lymphocytes to obtain a sample of cultured lymphocytes; and
infusing the sample to the patient,
wherein the time taken from acquiring the lymphocytes to infusing the sample is not longer than 28 days.

2. A method for preventing and/or reducing the likelihood of prolonged thrombocytopenia in a patient having r/r LBCL, the method comprising
acquiring lymphocytes from the patient through apheresis;
incubating lymphocytes with a polynucleotide vector to transduce the lymphocytes to produce transduced lymphocytes;
culturing the transduced lymphocytes to obtain a sample of cultured lymphocytes; and
infusing the sample to the patient,
wherein the time taken from acquiring the lymphocytes to infusing the sample is not longer than 28 days.

3. The method of claim 2, wherein the patient has
a greater than 55% likelihood of having complete response;
a greater than 45% likelihood of having overall survival at 24 months; and/or
a lower than 30% likelihood of developing prolonged thrombocytopenia.

4. The method of claim 2, wherein the time taken from acquiring the lymphocytes to infusing the sample is not longer than 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, or 6 days.

5. The method of claim 2, the method further comprising administering lymphodepleting chemotherapy, and wherein the lymphodepleting chemotherapy is administered within 5 days, 4 days, 3 days, 2 days, or 1 days of the infusing step.

6. The method of claim 2, which does not include cryopreservation of the cultured lymphocytes.

7. The method of claim 2, wherein the transduced lymphocytes are cultured for less than 72 hours, 48 hours or 36 hours.

8. The method of claim 2, wherein the incubation is carried out in a closed system, and wherein the closed system has an inner surface area of at least 1500 cm².

9. (canceled)

10. The method of claim 8, wherein the closed system has an inner surface coated with a recombinant human fibronectin, wherein the coating is carried out with a solution that comprises about 1-10 μg/ml of the recombinant human fibronectin.

11. The method of claim 10, wherein the inner surface is further in contact with a second solution comprising the polynucleotide vector, wherein the second solution has a volume of about 200 mL.

12. (canceled)

13. (canceled)

14. (canceled)

15. The method of claim 2, wherein the lymphocytes are peripheral blood mononuclear cells (PBMCs) or T cells.

16. (canceled)

17. The method of claim 2, wherein a total of 10,000 to 1,000,000 cultured lymphocytes per kilogram of the patient are administered to the patient.

18. The method of claim 17, wherein a total of 20,000 to 400,000 cultured lymphocytes per kilogram of the patient are administered to the patient.

19. The method of claim 18, wherein at least 15% of the cultured lymphocytes are transduced with the vector.

20. (canceled)

21. (canceled)

22. The method of claim 2, wherein the vector encodes one or more chimeric antigen receptors (CAR) or one or more T cell receptors (TCR).

23. (canceled)

24. (canceled)

25. (canceled)

26. (canceled)

27. (canceled)

28. (canceled)

29. (canceled)

30. (canceled)

31. A method for predicting a likelihood of a complete response in a patient to an immunotherapy, comprising:
determining a period of time from a leukapheresis step of said patient to an administration of said immunotherapy to said patient;
grouping said patient into one of a plurality of groups based on said determining a period of time, said plurality of groups comprising:
a first group characterized by a period of time of up to 28 days from said leukapheresis step to said administration of said immunotherapy to said patient;
a second group characterized by a period of time of between 28 days to 40 days from said leukapheresis step to said administration of said immunotherapy to said patient; and
a third group characterized by a period of time of at least 40 days from said leukapheresis step to said administration of said immunotherapy to said patient; and
determining said likelihood of a complete response in said patient based at least in part on which of said plurality of groups said patient is grouped into,
wherein said patient has at least about a 55% likelihood of a complete response if said patient is grouped within said first group or said second group, and
wherein said patient has at least about a 42% likelihood of a complete response if said patient is grouped within said third group.

32. (canceled)

33. (canceled)

34. (canceled)

35. (canceled)

36. (canceled)

37. (canceled)

38. A method for predicting an overall survival rate in a patient to an immunotherapy, comprising:
determining a period of time from a leukapheresis step of said patient to an administration of said immunotherapy to said patient;
grouping said patient into one of a plurality of groups based on said determining a period of time, said plurality of groups comprising:
a first group characterized by a period of time of up to 28 days from said leukapheresis step to said administration of said immunotherapy to said patient;
a second group characterized by a period of time of between 28 days to 40 days from said leukapheresis step to said administration of said immunotherapy to said patient; and
a third group characterized by a period of time of at least 40 days from said leukapheresis step to said administration of said immunotherapy to said patient; and
determining said overall survival rate in said patient based at least in part on which of said plurality of groups said patient is grouped into,
wherein said patient has at least about a 49% overall survival rate if said patient is grouped within said first group,
wherein said patient has at least about a 48% overall survival rate if said patient is grouped within said second group, and
wherein said patient has at least about a 30% overall survival rate if said patient is grouped within said third group.

39. (canceled)

40. (canceled)

41. (canceled)

42. (canceled)

43. (canceled)

44. A method for predicting a risk of thrombocytopenia in a patient receiving an immunotherapy, comprising:
determining a period of time from a leukapheresis step of said patient to an administration of said immunotherapy to said patient;
grouping said patient into one of a plurality of groups based on said determining a period of time, said plurality of groups comprising:
a first group characterized by a period of time of up to 28 days from said leukapheresis step to said administration of said immunotherapy to said patient;
a second group characterized by a period of time of between 28 days to 40 days from said leukapheresis step to said administration of said immunotherapy to said patient; and
a third group characterized by a period of time of at least 40 days from said leukapheresis step to said administration of said immunotherapy to said patient; and
determining said risk of thrombocytopenia in said patient based at least in part on which of said plurality of groups said patient is grouped into,
wherein said patient has about an 18% risk of thrombocytopenia if said patient is grouped within said first group,
wherein said patient has about a 25% risk of thrombocytopenia if said patient is grouped within said second group, and
wherein said patient has about a 34% risk of thrombocytopenia if said patient is grouped within said third group.

45. (canceled)

46. (canceled)

47. (canceled)

48. (canceled)

49. (canceled)

50. A method for predicting life expectancy and a quality-adjusted life years in a patient that has received an immunotherapy, comprising:
determining a period of time from a leukapheresis step of said patient to an administration of said immunotherapy to said patient, wherein the period of time is a short period or a long period;
assigning a probability of successful infusion based on the period of time;
entering the patient information into a survival model to determine the life expectancy and the quality-adjusted life years of the patient.

51. (canceled)

52. (canceled)

53. (canceled)

54. (canceled)

55. (canceled)

56. (canceled)

57. (canceled)

58. (canceled)

59. (canceled)


SEQUENCE LISTING (27 sequences)

SEQ ID NO: 1 - protein, 120 aa, synthetic construct
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARGGGSWYSNWFDPWGQGTMVTVSS

SEQ ID NO: 2 - protein, 107 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQDRSLPPTFGGGTKVEIK

SEQ ID NO: 3 - protein, 121 aa, synthetic construct
QVQLQQWGAGLLKPSETLSLTCAVYGGSFSGIHWNWIRQPPGKGLEWIGDIDTSGSTNYNPSLKSRVTISVDTSKNQFSLKLSSVTAADTAVYYCARLGQESATYLGMDVWGQGTTVTVSS

SEQ ID NO: 4 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQLYTYPFTFGGGTKVEIK

SEQ ID NO: 5 - protein, 124 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARETDYSSGMGYGMDVWGQGTTVTVSS

SEQ ID NO: 6 - protein, 107 aa, synthetic construct
DIQMTQSPSSLSASVGDRVTITCRASQSINSYLNWYQQKPGKAPKLLIYAASSLQSGVPSRFSGSGSGTDFTLTISSLQPEDFATYYCQQSLADPFTFGGGTKVEIK

SEQ ID NO: 7 - protein, 125 aa, synthetic construct
QVQLVQSGAEVKKPGASVKVSCKASGYTFKEYGISWVRQAPGQGLEWMGWISAYSGHTYYAQKLQGRVTMTTDTSTSTAYMELRSLRSDDTAVYYCARGPHYDDWSGFIIWFDPWGQGTLVTVSS

SEQ ID NO: 8 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYRFPPTFGQGTKVEIK

SEQ ID NO: 9 - protein, 124 aa, synthetic construct
QVQLQESGPGLVKPSETLSLTCTVSGGSISSPDHYWGWIRQPPGKGLEWIGSIYASGSTFYNPSLKSRVTISVDTSKNQFSLKLSSVTAADTAVYYCARETDYSSGMGYGMDVWGQGTTVTVSS

SEQ ID NO: 10 - protein, 107 aa, synthetic construct; identical to SEQ ID NO: 6

SEQ ID NO: 11 - protein, 123 aa, synthetic construct
QITLKESGPTLVKPTQTLTLTCTFSGFSLDTEGVGVGWIRQPPGKALEWLALIYFNDQKRYSPSLKSRLTITKDTSKNQVVLTMTNMDPVDTAVYYCARDTGYSRWYYGMDVWGQGTTVTVSS

SEQ ID NO: 12 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQAYAYPITFGGGTKVEIK

SEQ ID NO: 13 - protein, 127 aa, synthetic construct
QVQLQQWGAGLLKPSETLSLTCAVYGGSFEKYYWSWIRQPPGKGLEWIGEIYHSGLTNYNPSLKSRVTISVDTSKNQFSLKLSSVTAADTAVYYCARVRYDSSDSYYYSYDYGMDVWGQGTTVTVSS

SEQ ID NO: 14 - protein, 113 aa, synthetic construct
DIVLTQSPDSLAVSLGERATINCKSSQSVLYSSNNKNYLAWYQQKPGQPPKLLIYWASSRESGVPDRFSGSGSGTDFTLTISSLQAEDVAVYYCQQSYSFPWTFGGGTKVEIK

SEQ ID NO: 15 - protein, 127 aa, synthetic construct
QVQLQQWGAGLLKPSETLSLTCAVYGGSFSRYVWSWIRQPPGKGLEWIGEIDSSGKTNYNPSLKSRVTISVDTSKNQFSLKLSSVTAADTAVYYCARVRYDSSDSYYYSYDYGMDVWGQGTTVTVSS

SEQ ID NO: 16 - protein, 113 aa, synthetic construct; identical to SEQ ID NO: 14

SEQ ID NO: 17 - protein, 127 aa, synthetic construct
QVQLQQWGAGLLKPSETLSLTCAVYGGSFSGYAWSWIRQPPGKGLEWIGEIDHRGFTNYNPSLKSRVTISVDTSKNQFSLKLSSVTAADTAVYYCARVRYDSSDSYYYSYDYGMDVWGQGTTVTVSS

SEQ ID NO: 18 - protein, 113 aa, synthetic construct; identical to SEQ ID NO: 14

SEQ ID NO: 19 - protein, 123 aa, synthetic construct
QVQLQQWGAGLLKPSETLSLTCAVYGGSFQKYYWSWIRQPPGKGLEWIGEIDTSGFTNYNPSLKSRVTISVDTSKNQFSLKLSSVTAADTAVYYCARVGRYSYGYYITAFDIWGQGTTVTVSS

SEQ ID NO: 20 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQHYSFPFTFGGGTKVEIK

SEQ ID NO: 21 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 22 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 23 - protein, 120 aa, synthetic construct
EVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSS

SEQ ID NO: 24 - protein, 107 aa, synthetic construct
DIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIK

SEQ ID NO: 25 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 26 - protein, 1018 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRSGSGEGRGSLLTCGDVEENPGPMALPVTALLLPLALLLHAARPQLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARETDYSSGMGYGMDVWGQGTTVTVSSGSTSGSGKPGSGEGSTKGDIQMTQSPSSLSASVGDRVTITCRASQSINSYLNWYQQKPGKAPKLLIYAASSLQSGVPSRFSGSGSGTDFTLTISSLQPEDFATYYCQQSLADPFTFGGGTKVEIKAAAFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 733 aa, synthetic construct
MLLLVTSLLLCELPHPAFLLIPDIQMTQSPSSLSASVGDRVTITCRASQSINSYLNWYQQKPGKAPKLLIYAASSLQSGVPSRFSGSGSGTDFTLTISSLQPEDFATYYCQQSLADPFTFGGGTKVEIKGGGGSGKPGSGEGGSQLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARETDYSSGMGYGMDVWGQGTTVTVSSGGGGSGKPGSDIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIKGGGGSGGGGSGGGGSEVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR